SEQUENCE LISTING (27 sequences)

SEQ ID NO: 1 - protein, 331 aa, Fusarium oxysporum
SPVALDDYVNSLEERAVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRRYRSAESVDKRATMTDAELEKKLNSYVQMDKEYVKNNQARS

SEQ ID NO: 2 - protein, 92 aa, Artificial Sequence
  Hybrid signal sequence
MRQVWFSWIVGLFLCFFNVSSAAPVNTTTEDETAQIPAEAVIGYSDLEGDFDVAVLPFSNSTNNGLLFINTTIASIAAKEEGVSLEKREAEA

SEQ ID NO: 3 - protein, 15 aa, Fusarium oxysporum
MLLLPLLSAITLAVA

SEQ ID NO: 4 - protein, 316 aa, Fusarium oxysporum
AVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRRYRSAESVDKRATMTDAELEKKLNSYVQMDKEYVKNNQARS

SEQ ID NO: 5 - protein, 19 aa, Saccharomyces cerevisiae
MLLQAFLFLLAGFAAKISA

SEQ ID NO: 6 - protein, 28 aa, Saccharomyces cerevisiae
MKFSAGAVLSWSSLLLASSVFAQQEAVA

SEQ ID NO: 7 - protein, 26 aa, Saccharomyces cerevisiae
MTKPTQVLVRSVSILFFITLLHLVVA

SEQ ID NO: 8 - protein, 19 aa, Saccharomyces cerevisiae
MRFPSIFTAVLFAASSALA

SEQ ID NO: 9 - protein, 30 aa, Fusarium oxysporum
MLLLPLLSAITLAVASPVALDDYVNSLEER

SEQ ID NO: 10 - protein, 5 aa, Artificial Sequence
  linker sequence
GGGGS

SEQ ID NO: 11 - protein, 15 aa, Artificial Sequence
  Linker sequence
GGGGSGGGGSGGGGS

SEQ ID NO: 12 - protein, 8 aa, Artificial Sequence
  linker sequence
GGGGGGGG

SEQ ID NO: 13 - protein, 40 aa, Artificial Sequence
  linker sequence
GGGGSGGGGSGGGGSGGGGSGGGGSGGGGSGGGGSGGGGS

SEQ ID NO: 14 - protein, 12 aa, Artificial Sequence
  linker sequence
GSAGSAAGSGEF

SEQ ID NO: 15 - protein, 4 aa, Artificial Sequence
  linker sequence
EAAK

SEQ ID NO: 16 - protein, 5 aa, Artificial Sequence
  linker sequence
EAAAK

SEQ ID NO: 17 - protein, 12 aa, Artificial Sequence
  linker sequence
EAAKEAAKEAAK

SEQ ID NO: 18 - protein, 46 aa, Artificial Sequence
  linker sequence
AEAAAKEAAAKEAAAKEAAAKALEAEAAAKEAAAKEAAAKEAAAKA

SEQ ID NO: 19 - protein, 20 aa, Artificial Sequence
  linker sequence
APAPAPAPAPAPAPAPAPAP

SEQ ID NO: 20 - protein, 22 aa, Artificial Sequence
  linker sequence
KDNSSTIEGRYPYDVPDYALQA

SEQ ID NO: 21 - protein, 490 aa, Artificial Sequence
  Synthetic construct
FPVRRTDLGQVQGLAGDVISFRGIPYAAPPVGDLRWKPPQHARPWAGVRPATQFGSDCFGAAYLRKGSLAPGVSEDCLYLNVWAPSDAKPGQYPVMVWVYGGGFAGGTAAMPYYDGEALARQGVVVVTFNYRTNIFGFFAHPGLSRESPTGTSGNYGLLDILAALRWVQSNARAFGGDPGRVTVFGESAGASAIGLLLTSPLSKGLFRGAILESPGLTRPLATLADSAASGERLDADLSRLRSTDPATLMARADAARPASRDLRKPRPIGPIVDGHVLPQTDSDAIAAGQLAPVPVLIGTNADEGRAFLGRAPIETPADYQAYLEALFGDQAAAVAACYPLDGRATPKEMVARIFGDNQFNRGVSAFSEALVRHGVPVWRYQFIGNTEDGRAPATHGAEIPYVFGVFKLDELGLFDWPPEGPTLADRALCQLMSSAWVRFAKNGDPAGDALTWPAYSTGKSTMTFGPEGRAAVVSPGPSIPSCADGVKAG

SEQ ID NO: 22 - protein, 597 aa, Artificial Sequence
  Synthetic construct
  SIGNAL (1)...(19)
MRFPSIFTAVLFAASSALAETLENGGVIMQAFYWDVPGGGIWNDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVGAEAAAKALEAEAAAKEAAAKEAAAKEAAAKAAANVTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 23 - protein, 432 aa, Thermococcus hydrothermalis
ETLENGGVIMQAFYWDVPGGGIWNDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 24 - protein, 19 aa, Saccharomyces cerevisiae
MRFPSIFTAVLFAASSALA

SEQ ID NO: 25 - protein, 34 aa, Artificial Sequence
  Synthetic construct
AEAAAKALEAEAAAKEAAAKEAAAKEAAAKAAAN

SEQ ID NO: 26 - protein, 112 aa, Saccharomyces cerevisiae
VTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 27 - protein, 529 aa, Saccharomyces cerevisiae
MKFSAGAVLSWSSLLLASSVFAQQEAVAPEDSAVVKLATDSFNEYIQSHDLVLAEFFAPWCGHCKNMAPEYVKAAETLVEKNITLAQIDCTENQDLCMEHNIPGFPSLKIFKNSDVNNSIDYEGPRTAEAIVQFMIKQSQPAVAVVADLPAYLANETFVTPVIVQSGKIDADFNATFYSMANKHFNDYDFVSAENADDDFKLSIYLPSAMDEPVVYNGKKADIADADVFEKWLQVEALPYFGEIDGSVFAQYVESGLPLGYLFYNDEEELEEYKPLFTELAKKNRGLMNFVSIDARKFGRHAGNLNMKEQFPLFAIHDMTEDLKYGLPQLSEEAFDELSDKIVLESKAIESLVKDFLKGDASPIVKSQEIFENQDSSVFQLVGKNHDEIVNDPKKDVLVLYYAPWCGHCKRLAPTYQELADTYANATSDVLIAKLDHTENDVRGVVIEGYPTIVLYPGGKKSESVVYQGSRSLDSLFDFIKENGHFDVDGKALYEEAQEKAAEEAEADAEAEADADAELADEEDAIHDE

(19) United States
(12) Patent Application Publication
(10) Pub. No.: US 2024/0084244 A1
(43) Pub. Date: Mar. 14, 2024

(54) RECOMBINANT YEAST HOST CELL HAVING ENHANCED GROWTH RATE

(71) Applicant: DANSTAR FERMENT AG, Zug (CH)

(72) Inventors: Zhiqing WANG, Lebanon, NH (US); Aditi RANADE, State College, PA (US); Aaron ARGYROS, Lebanon, NH (US); Allan FROEHLICH, Lebanon, NH (US); Charles F. RICE, Lebanon, NH (US)

(21) Appl. No.: 18/262,336

(22) PCT Filed: Jan. 26, 2022
(86) PCT No.: PCT/IB2022/050686

§ 371 Date: Jul. 20, 2023

Related U.S. Application Data
(60) Provisional application No. 63/141,807, filed on Jan. 26, 2021.

(51) Int. Cl. C12N 1/18; C12N 9/18; C12N 9/28

(52) U.S. Cl. CPC C12N 1/185; C12N 9/18; C12N 9/2417; C12R 2001/865; C12Y 301/01004; C12Y 301/01087; C12Y 302/01001

(57) ABSTRACT

The present disclosure concerns recombinant yeast host cells having a first genetic modification to express a heterologous polypeptide or to over-express a native polypeptide. The recombinant yeast host cells also have a second genetic modification to at least partially mitigate the reduction in growth rate resulting from the expression of the heterologous polypeptide or the over-expression of the native polypeptide. The second genetic modification can be, for example, to favor the secretion of the heterologous or native polypeptide.

### CROSS-REFERENCE TO RELATED APPLICATION(S) AND DOCUMENT(S)

The present application claims priority from U.S. provisional application 63/141,807 filed on Jan. 26, 2021 and herewith incorporated herewith in its entirety. The present application also includes a sequence listing in electronic format also incorporated herewith in its entirety.

### TECHNOLOGICAL FIELD

The present disclosure relates to a recombinant yeast host cell that has been modified to restore at least in part its growth rate from a growth defect resulting from the expression of a heterologous polypeptide or from the over-expression of a native polypeptide.

### BACKGROUND

Polypeptides, especially enzymes, have applications in various industries, and the most significant industrial applications can be used, for example, in the food, detergent, and pharmaceutical industries. Limitations on the industrial use of these proteins (which can be enzymes) have largely been due to their high production costs. In bread making, chemical emulsifiers, such as SSL and DATEM, are widely used to strengthen the dough, to increase the bread volume, to soften the crumb, and to delay bread stalling. Enzymes are considered as clean label improvers, and depending on the specific enzyme, they are often assumed to be denatured or deactivated during baking, thus having no remaining activity in the final products. Lipases and phospholipases, therefore, offer the opportunity to reduce or replace chemical emulsifiers in bread making.

However, production of heterologous polypeptides from recombinant expression has a number of challenges. Overexpression of phospholipase has been reported to cause growth impairment in several production organisms, due to its activity on phospholipids of membrane components. When expressing phospholipase using a yeast host cell, the enzyme can be targeted for secretion out of the cell, where the cell wall of yeast acts as a diffusion barrier to block the access of phospholipase to its cytoplasmic membrane, which is one of the targets of phospholipase. However, toxicity is still observed as the enzyme intended to be secreted can still have activity before it is fully transported out of the yeast host cell. This results in toxicity and reduced growth rate of the yeast host cell, which in turn lowers the production titer of lipases and phospholipases by the host cell.

As such, improved recombinant host cells and processes are needed to reduce toxicity and restore, at least in part, the growth rate of the host cells expressing heterologous polypeptides or overexpressing native polypeptides.

### SUMMARY

The present disclosure provides recombinant yeast host cells which have been genetically engineered to express a heterologous polypeptide or overexpress a native polypeptide as well as a genetic modification to restore, at least in part, their growth rate.

According to a first aspect, the present disclosure provides a recombinant yeast host cell having (i) a first genetic modification for (a) expressing a heterologous polypeptide or over-expressing a native polypeptide and (b) a signal sequence operatively associated with the heterologous polypeptide or the native polypeptide; and (ii) a second genetic modification for increasing the growth rate of the recombinant yeast host cell when compared to the growth rate of a control yeast host cell. The control yeast host cell expresses the heterologous polypeptide or over- expresses the native polypeptide and lacks the second genetic modification. The expression of the heterologous polypeptide or the over-expression of the native polypeptide impedes the growth rate of the control yeast host cell when compared to a parental yeast host cell (lacking both the first and the second genetic modification) or to the recombinant yeast host cell (having both the first and the second genetic modification. In an embodiment, the recombinant yeast host cell comprises a first heterologous nucleic acid molecule encoding the heterologous polypeptide or a first native nucleic acid molecule encoding the native polypeptide. In another embodiment, the first heterologous nucleic acid molecule or the first native nucleic acid molecule is operatively associated with a propagation or an aerobic promoter. In still another embodiment, the heterologous polypeptide or the native polypeptide is secreted, and, in further embodiment, is tethered. In some embodiments, the heterologous or native polypeptide is a polypeptide having phospholipase activity. In further embodiments, the polypeptide having phospholipase activity comprises an amino acid sequence of SEQ ID NO. 1 or 4, is a variant of the amino acid sequence of SEQ ID NO: 1 or 4 having phospholipase activity, or is a fragment of the amino acid sequence of SEQ ID NO: 1 or 4 having phospholipase activity. In other embodiments, the heterologous or native polypeptide is a polypeptide having fumonisin esterase activity. In further embodiments, the polypeptide having fumonisin esterase activity comprises an amino acid sequence of SEQ ID NO: 21, is a variant of the amino acid sequence of SEQ ID NO: 21 having fumonisin esterase activity, or is a fragment of the amino acid sequence of SEQ ID NO: 21 having fumonisin esterase activity. In other embodiments, the heterologous or the native polypeptide is a polypeptide having alpha-amylase activity. In other embodiments, the polypeptide having alpha-amylase activity comprises an amino acid sequence of SEQ ID NO: 23, is a variant of the amino acid sequence of SEQ ID NO: 23 having alpha-amylase activity, or is a fragment of the amino acid sequence of SEQ ID NO: 23 having alpha-amylase activity. In yet another embodiment, the signal sequence comprises an amino acid sequence of SEQ ID NO: 2, 3 or 24, is a variant of the amino acid sequence of SEQ ID NO: 2, 3 or 24 or is a fragment of SEQ ID NO: 2, 3 or 24. In an embodiment, the second genetic modification is a modification in a yeast protein secretory and trafficking pathway. For example, the second genetic modification can be one or more of a modification of a yeast translation pathway; one or more of a modification of a yeast post-translational modification pathway; one or more of modification of a yeast protein trafficking pathway; one or more of modification of a yeast vacuolar pathway; one or more of a modification of a yeast cell wall stability pathway; or combinations thereof. In another embodiment, the second genetic modification comprises a modulation in the expression of a gene involved in polypeptide folding, glycosylation and degradation in the endoplasmic reticulum (ER). In a further embodiment, the second genetic modification comprises the modulation in the expression of a gene associated with a molecular chaperone. For example, the gene associated with the molecular chaperone can comprise a KAR2 gene, a LHS1 gene, a JEM1 gene, a SSA1 gene, a SSA4 gene and/or a SSE1 gene. In a further embodiment, the second genetic modification comprises the modulation in the expression of a gene associated with an unfolded protein response (UPR). For example, the gene associated with UPR can comprises a HAC1 gene, an IRE1 gene, and/or a KIN2 gene. In a further embodiment, the second genetic modification comprises the modulation in the expression of a gene associated with a redox enzyme. For example, the gene associated with the redox enzyme can comprise a PDI1 gene, an ERO1 gene, and/or a CPR5 gene. In a specific embodiment, the gene associated with the redox enzyme is a PDI1 gene and in yet another embodiment, the second genetic modification can be the overexpression of the PD1 gene. In a specific embodiment, the gene associated with the redox enzyme is a ERO1 gene and in yet another embodiment, the second genetic modification can be the overexpression of the ERO1 gene. In a specific embodiments, the genes associated with the redox enzyme are a PDI1 gene and a ERO1 gene and in yet another embodiment, the second genetic modification can be the overexpression of the PDI1 and the ERO1 genes. In a further embodiment, the second genetic modification comprises the modulation of the expression of a gene involved in endoplasmic reticulum associated degradation pathway (ERAD). For example, the gene involved in ERAD can comprise a HTM1 gene, a YOS9 gene, a HDR1 gene, a HDR3 gene, an UBC7 gene, and/or a DER1 gene. In a further embodiment, the second genetic modification comprises the modulation in the expression of a gene involved in endoplasmic reticulum (ER) expansion. For example, the gene involved in ER expansion can comprise an OPI1 gene, and/or a PAH1 gene. In a further embodiment, the second genetic modification comprises the modulation in the expression of a gene involved in translocation to the endoplasmic reticulum (ER). For example, the gene involved in translocation to the ER can comprise a SIL1 gene. In another embodiment, the second genetic modification comprises the modulation in the expression of a gene involved in polypeptide trafficking from the ER to the Golgi system, and/or from the Golgi system to the plasma membrane. In a further embodiment, the second genetic modification comprises the modulation in the expression of a gene associated with a COPII vesicle. For example, the gene associated with the COPII vesicle can comprise an ERV25 gene, an ERV29 gene, a SAR1 gene, a SEC12 gene, a SEC13 gene, a SEC16 gene, a SEC23 gene, a SE24 gene, a SEC31 gene, a SBH1 gene, COG5 gene, a COG6 gene, a BOS1 gene, COY1 gene, and/or a SLY1 gene. In a further embodiment, the second genetic modification comprises the modulation in the expression of a gene associated with a COPI vesicle. For example, the gene associated with the COPI vesicle can comprise a GOS1 gene, and/or a LAM1 gene. In a further embodiment, the second genetic modification comprises the modulation in the expression of a gene associated with an endosome. For example, the gene associated with the endosome can comprise a VPS5 gene, a VPS17 gene, a VPS26 gene, a VPS29 gene, and/or a VPS35 gene. In a specific embodiment, the gene associated with endosomes is a VPS5 gene and in yet a further specific embodiment, the second genetic modification is the inactivation of the VPS5 gene. In a specific embodiment, the gene associated with endosomes is a VPS17 gene and in yet a further specific embodiment, the second genetic modification is the inactivation of the VPS17 gene. In a further embodiment, the second genetic modification comprises the modulation in the expression of a gene involved in polypeptide trafficking from the Golgi system to the plasma membrane. For example, the gene involved in polypeptide trafficking from the Golgi system to the plasma membrane can comprise a SEC1 gene, a SEC4 gene, a SSO1 gene, a SSO2 gene, a SNC2 gene, an EX070 gene, and/or a YPT32 gene. In another embodiment, the second genetic modification comprises the modulation in the expression of a gene involved in the vacuolar sorting pathway. In a further embodiment, the second genetic modification comprises the modulation in the expression of a gene associated with a vacuolar protease. For example, the gene associated with the vacuolar protease can comprise a PRB1 gene, a VMA3 gene, and/or a PEP4 gene. In a specific embodiment, the gene associated with the vacuolar protease is a PRB1 gene and in yet a further specific embodiment, the second genetic modification can be the inactivation of the PRB1 gene. In a further embodiment, the second genetic modification comprises the modulation in the expression of a gene associated with a polypeptide trafficking toward vacuoles. For example, the gene associated with the polypeptide trafficking toward vacuoles can comprise a VPS8 gene, and/or a VPS21 gene. In a further embodiment, the second genetic modification comprises the modulation in the expression of a gene involved in autophagy. For example, the gene involved in autophagy can comprise a MTC6 gene, and/or a SEC4 gene. In another embodiment, the second genetic modification comprises the modulation in the expression of a gene associated with a cell wall mannoprotein. For example, the gene associated with the cell wall mannoprotein can comprise a CCW12 gene, and/or a CWP2 gene. In another embodiment, the second genetic modification comprises the modulation in the expression of a gene associated with a cell wall glycoprotein. For example, the gene encoding the cell wall glycoprotein can comprise a SED1 gene. In another embodiment, the second genetic modification comprises the modulation in the expression of a gene involved in transcription and/or translation. For example, the gene involved in transcription and/or translation can comprise a BMH2 gene, a HSF1 gene, a SRP14 gene, and/or a SRP54 gene. In another embodiment, the second genetic modification comprises the modulation in the expression of a gene associated with a ribosome. For example, the gene associated with the ribosome can comprise a BFR2 gene, and/or a RPP0 gene. In another embodiment, the second genetic modification comprises the modulation in the expression of a gene associated with a protease involved in the activation of proproteins of the secretory pathway. For example, the gene associated with the protease involved in the activation of proproteins of the secretory pathway can comprise a KEX2 gene. In a specific embodiment, the second genetic modification is in the KEX2 gene and in further embodiments, the second genetic modification is the overexpression of the KEX2 gene. In an embodiment, the recombinant yeast host cell is from the genus Saccharomyces sp. In another embodiment, the recombinant yeast host cell is from the species Saccharomyces cerevisiae.

According to a second aspect, the present disclosure provides a process for propagating the recombinant yeast host cell described herein, the process comprising culturing the recombinant yeast host cell in a culture medium under conditions so as to allow the propagation of the recombinant yeast host cell. In another embodiment, the process is aerobic. In some further embodiments, the process further comprises isolating the heterologous or native polypeptide from the culture medium. In another embodiment, the process further comprises isolating the heterologous or native polypeptide from the propagated recombinant yeast host cells. In an embodiment, the process further comprises providing the recombinant yeast host cell. In still another embodiment, the process further comprises introducing, in a parental yeast host cell, the first and the second genetic modification defined herein.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1A shows the growth curves of a wild-type Saccharomyces cerevisiae stain (solid line), a recombinant Saccharomyces cerevisiae stain expressing heterologous phospholipase (dotted line) and a recombinant Saccharomyces cerevisiae stain expressing heterologous phospholipase and having PRB1 deletion (prb1Δ, dashed line). X-axis indicate time in minutes, Y-axis indicate cell density as measured by optical density at 600 nm (OD600).

FIG. 1B shows a graph comparing the phospholipase activity in the culture supernatant of recombinant Saccharomyces cerevisiae stain expressing a heterologous phospholipase (wild-type) and recombinant Saccharomyces cerevisiae stain expressing a heterologous phospholipase and having PRB1 deletion (ΔPRB1). X-axis indicate stain type, Y-axis indicate phospholipase activity (ΔRFU at excitation/emission of 488/530 nm).

FIG. 2A shows the growth curves of a wild-type Saccharomyces cerevisiae stain (solid line), a recombinant Saccharomyces cerevisiae stain expressing a heterologous phospholipase (dotted line), a recombinant Saccharomyces cerevisiae stain expressing a heterologous phospholipase and having VPS5 deletion (dashed line), and a recombinant Saccharomyces cerevisiae stain expressing a heterologous phospholipase and having VPS17 deletion (Δ). X-axis indicate time in minutes, Y-axis indicate density as measured by optical density at 600 nm (OD600).

FIG. 2B shows a graph comparing the phospholipase activity in the culture supernatant of a recombinant Saccharomyces cerevisiae stain expressing a heterologous phospholipase (Wild-type), a recombinant Saccharomyces cerevisiae stain expressing a heterologous phospholipase and having VPS5 deletion (ΔVps5), and a recombinant Saccharomyces cerevisiae stain expressing heterologous phospholipase and having VPS17 deletion (ΔVps17). X-axis indicate stain type, Y-axis indicate phospholipase activity (ΔRFU at excitation/emission of 488/530 nm).

FIG. 3A shows the growth curves of a wild-type Saccharomyces cerevisiae stain (solid line), a recombinant Saccharomyces cerevisiae stain expressing a heterologous phospholipase (dotted line), a recombinant Saccharomyces cerevisiae stain expressing a heterologous phospholipase and having PRB1 deletion (dashed line), a recombinant Saccharomyces cerevisiae stain expressing a heterologous phospholipase and having simultaneous PDI1 and ERO1 overexpression (“X”), and a recombinant Saccharomyces cerevisiae stain expressing a heterologous phospholipase and having simultaneous PDI1 and ERO1 overexpression in addition to PRB1 deletion (“□”). X-axis indicate time in minutes, Y-axis indicate cell density as measured by optical density at 600 nm (OD600).

FIG. 3B shows a graph comparing the phospholipase activity in the culture supernatant of a recombinant Saccharomyces cerevisiae stain expressing a heterologous phospholipase and having PRB1 deletion (ΔPRB1), and a recombinant Saccharomyces cerevisiae stain expressing heterologous phospholipase and having simultaneous PDI1 and ERO1 overexpression in addition to PRB1 deletion (ΔPRB1 Pdi1+Ero1 Overexpression). X-axis indicate stain type, Y-axis indicate phospholipase activity (ΔRFU at excitation/emission of 488/530 nm).

FIG. 4 compares the maximal growth rate (μMax in h⁻¹) of S. cerevisiae strain M10580 and transformants T11203 and T11204.

FIG. 5 compares the combined secreted and cell associated Ceralpha activity (as measured as the optical density at 400 nm) of S. cerevisiae strain M10580 and transformants T11203 and T11204.

### DETAILED DESCRIPTION

The present disclosure provides recombinant yeast host cells a first genetic modification to express a heterologous polypeptide or to over-express a native polypeptide. The heterologous polypeptide or the native polypeptide intended to be expressed or over-expressed in the recombinant yeast host cell is intended to be secreted/exported. However, the expression of the heterologous polypeptide or the over-expression of the native polypeptide results in a reduction of growth rate (and in some embodiments of the maximal growth rate) of a control yeast host cell expressing same. This reduction in the growth rate can be observed in a control yeast host cell (which is capable of expressing the heterologous polypeptide or over-expressing the native polypeptide) when compared to its parental yeast host cell. As used in the context of the present application, a “parental yeast host cell” is cell that does not include the first and the second genetic modifications as described herein. The parental yeast host cell can be used to obtain the control yeast host cell and/or the recombinant yeast host cell. In some embodiments, the parental yeast host cells corresponds to the control yeast host cell, except that it lacks the first genetic modification(s) present in the control yeast host cell. In some further embodiments, the parental yeast host cells corresponds to the recombinant yeast host cell, except that it lacks the first and second genetic modifications present in the recombinant yeast host cell. In some embodiments, this reduction in the growth rate can also be observed in the control yeast host cell when compared to the recombinant yeast host cell of the present disclosure (which is capable of expressing the heterologous polypeptide or over-expressing the native polypeptide and including a further (e.g., second) genetic modification as described herein). The recombinant yeast host cell described herein comprises a second genetic modification (which is absent in the control yeast host cell and the parental yeast host cell) to overcome or at least partially mitigate the reduction in growth rate (e.g., the reduction in maximal growth rate) caused by the first genetic modification (in the control yeast host cell).

The recombinant yeast host cell of the present disclosure comprises at least two distinct genetic modifications. These recombinant yeast host cells can be obtained by introducing a first and a second genetic modifications (in any order) in a corresponding parental yeast host cell. When the genetic modification is aimed at increasing the expression of a specific targeted gene (which may native or heterologous), the genetic modification can be made in one or multiple genetic locations. When the genetic modification is aimed at reducing or inhibiting the expression of a specific targeted gene (which is endogenous to the host cell), the genetic modifications can be made in one or all copies of the targeted gene(s). In the context of the present disclosure, when recombinant yeast host cells are qualified as being “genetically engineered”, it is understood to mean that they have been manipulated to either add at least one or more heterologous or exogenous nucleic acid residue and/or remove at least one endogenous (or native) nucleic acid residue. In some embodiments, the one or more nucleic acid residues that are added can be derived from an heterologous cell or the recombinant cell itself. In the latter scenario, the nucleic acid residue(s) is (are) added at a genomic location which is different than the native genomic location. The genetic manipulations did not occur in nature and are the results of in vitro manipulations of the native yeast or bacterial host cell.

In some embodiments, each genetic modification can be encoded on one or more heterologous or native nucleic acid molecules. In some embodiments, the heterologous or native nucleic acid molecule can encode one or more polypeptide (which may be additional copies of a native gene).

In other embodiments, the heterologous nucleic acid molecules can encode one or more promoters or other regulatory sequences for modulating (e.g., increasing or decreasing) the expression of the native polypeptide. In some embodiments, the heterologous nucleic acid molecules of the present disclosure can include a signal sequence to favor the secretion of the heterologous polypeptide or the native polypeptide intended to be expressed or over-expressed by the recombinant yeast host cell.

The term “heterologous” when used in reference to a nucleic acid molecule (such as a promoter, a terminator or a coding sequence) or a protein/polypeptide refers to a nucleic acid molecule or a protein/polypeptide that is not natively found in the recombinant host cell. “Heterologous” also includes a native coding region/promoter/terminator, or portion thereof, that was removed from the source organism and subsequently reintroduced into the source organism in a form that is different from the corresponding native gene. In some embodiments, a heterologous nucleic acid sequence is a copy of a native nucleic acid sequence that is introduced not in its natural location in the organism's genome. The heterologous nucleic acid molecule is purposively introduced into the recombinant yeast host cell. A heterologous element could be derived from a different strain of host cell, or from an organism of a different taxonomic group (e.g., different kingdom, phylum, class, order, family genus, or species, or any subgroup within one of these classifications). As used herein, the term “native” when used in inference to a gene, polypeptide, enzymatic activity, or pathway refers to an unmodified gene, polypeptide, enzymatic activity, or pathway originally found in the recombinant host cell. In some embodiments, heterologous polypeptides derived from a different strain of host cell, or from an organism of a different taxonomic group (e.g., different kingdom, phylum, class, order, family genus, or species, or any subgroup within one of these classifications) can be used in the context of the present disclosure.

The heterologous nucleic acid molecules of the present disclosure comprise a coding region for the heterologous polypeptide. A DNA or RNA “coding region” is a DNA or RNA molecule (preferably a DNA molecule) which is transcribed and/or translated into a heterologous polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, transcription terminators, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell (such as the recombinant yeast host cell of the present disclosure), a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a recombinant host cell. In eukaryotic cells, polyadenylation signals are considered control regions.

In some embodiments, the heterologous nucleic acid molecules of the present disclosure include a coding sequence for a heterologous polypeptide, optionally in combination with a promoter and/or a terminator. In some embodiments, the heterologous nucleic acid molecules of the present disclosure include a nucleic acid sequence encoding a promoter for overexpressing a native gene encoding a native polypeptide. In the heterologous nucleic acid molecules of the present disclosure, the promoter and the terminator (when present) are operatively linked to the nucleic acid coding sequence of the heterologous or native polypeptide, e.g., they control the expression and the termination of expression of the nucleic acid sequence of the heterologous or the native polypeptide. The heterologous nucleic acid molecules of the present disclosure can also include a nucleic acid sequence coding fora signal sequence, e.g., a short peptide sequence for exporting the heterologous polypeptide outside the host cell. When present, the nucleic acid sequence coding for the signal sequence is directly located upstream and in frame of the nucleic acid sequence coding for the heterologous polypeptide or the native polypeptide.

In the recombinant yeast host cell described herein, the nucleic acid molecule coding for the promoter and the nucleic acid molecule coding for the heterologous or the native polypeptide are operatively linked to one another. In the context of the present disclosure, the expressions “operatively linked” or “operatively associated” refers to fact that the promoter is physically associated to the nucleotide acid molecule coding for the heterologous or the native polypeptide in a manner that allows, under certain conditions, for expression of the heterologous polypeptide from the nucleic acid molecule. In an embodiment, the promoter can be located upstream (5′) of the nucleic acid sequence coding for the heterologous protein. In still another embodiment, the promoter can be located downstream (3′) of the nucleic acid sequence coding for the heterologous protein. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous or native polypeptide. The promoters can be located, in view of the nucleic acid molecule coding for the heterologous or native polypeptide, upstream, downstream as well as both upstream and downstream.

The term “promoter” refers to a DNA fragment capable of controlling the expression of a coding sequence or functional RNA. The term “expression,” as used herein, refers to the transcription and stable accumulation of sense mRNA from the heterologous nucleic acid molecule or the native gene described herein. Expression may also refer to translation of mRNA into a polypeptide. Promoters may be derived in their entirety from the promoter of a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. Promoters which cause a gene to be expressed during the propagation phase of a yeast cell are herein referred to as “propagation promoters”. Propagation promoters include both constitutive and inducible promoters, such as, for example, glucose-regulated, molasses-regulated, stress-response promoters (including osmotic stress response promoters) and aerobic-regulated promoters. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be native or heterologous to the nucleic acid molecule encoding the native or the heterologous polypeptide. The promoter can be heterologous to the native gene encoding the native polypeptide to be overexpressed. The promoter can be heterologous or derived from a strain being from the same genus or species as the recombinant host cell. In an embodiment, the promoter is derived from the same genus or species of the yeast host cell and the heterologous polypeptide is derived from a different genus than the host cell. The promoter can be a single promotor or a combination of different promoters. In some embodiments, the promoter is a propagation promoter. In some embodiments, the promoter is an aerobic promoter.

In the context of the present disclosure, the promoter controlling the expression of the heterologous polypeptide or the native polypeptide can be a constitutive promoter (such as, for example, tef2p (e.g., the promoter of the tef2 gene), cwp2p (e.g., the promoter of the cwp2 gene), ssa1p (e.g., the promoter of the ssa1 gene), eno1p (e.g., the promoter of the enol gene), hxk1 (e.g., the promoter of the hxk1 gene) and pgk1p (e.g., the promoter of the pgk1 gene). In some embodiment, the promoter is adh1p (e.g., the promoter of the adh1 gene). However, in some embodiments, it is preferable to limit the expression of the polypeptide. As such, the promoter controlling the expression of the heterologous polypeptide or the native polypeptide can be an inducible or modulated promoters such as, for example, a glucose-regulated promoter (e.g., the promoter of the hxt7 gene (referred to as hxt7p)) or a sulfite-regulated promoter (e.g., the promoter of the gpd2 gene (referred to as gpd2p or the promoter of the fzf1 gene (referred to as the fzf1p)), the promoter of the ssu1 gene (referred to as ssu1p), the promoter of the ssu1-r gene (referred to as ssur1-rp). In an embodiment, the promoter is an anaerobic-regulated promoters, such as, for example tdh1p (e.g., the promoter of the tdh1 gene), pau5p (e.g., the promoter of the pau5 gene), hor7p (e.g., the promoter of the hor7 gene), adh1p (e.g., the promoter of the adh1 gene), tdh2p (e.g., the promoter of the tdh2 gene), tdh3p (e.g., the promoter of the tdh3 gene), gpd1p (e.g., the promoter of the gdp1 gene), cdc19p (e.g., the promoter of the cdc19 gene), eno2p (e.g., the promoter of the eno2 gene), pdc1p (e.g., the promoter of the pdc1 gene), hxt3p (e.g., the promoter of the hxt3 gene), dan1 (e.g., the promoter of the dan1 gene) and tpi1p (e.g., the promoter of the tpi1 gene). One or more promoters can be used to allow the expression of each heterologous polypeptides in the recombinant yeast host cell.

One or more promoters can be used to allow the expression of each heterologous/native polypeptides in the recombinant yeast host cell. In the context of the present disclosure, the expression “functional fragment of a promoter” when used in combination to a promoter refers to a shorter nucleic acid sequence than the native promoter which retain the ability to control the expression of the nucleic acid sequence encoding the heterologous polypeptide. Usually, functional fragments are either 5′ and/or 3′ truncation of one or more nucleic acid residue from the native promoter nucleic acid sequence.

The heterologous nucleic acid molecule of the present disclosure can be integrated in the chromosome(s) of the yeast host cell. The term “integrated” as used herein refers to genetic elements that are placed, through molecular biology techniques, into the chromosome of a host cell. For example, genetic elements can be placed into the chromosomes of the host cell as opposed to in a vector such as a plasmid carried by the host cell. Methods for integrating genetic elements into the chromosome of a host cell are well known in the art and include homologous recombination. The heterologous nucleic acid molecule can be present in one or more copies in the yeast host cell's chromosome. Alternatively, the heterologous nucleic acid molecule can be independently replicating from the yeast's chromosome. In such embodiment, the nucleic acid molecule can be stable and self-replicating. The heterologous nucleic acid molecules can be present in one or more copies in the recombinant yeast host cell. For example, each heterologous nucleic acid molecules can be present in 1, 2, 3, 4, 5, 6, 7, 8 copies or more per chromosome.

The present disclosure also provides nucleic acid molecules for modifying the yeast host cell so as to allow the expression of the one or more heterologous polypeptide, variants or fragments thereof or the overexpression of one or more native polypeptide. The nucleic acid molecule may be DNA (such as complementary DNA, synthetic DNA or genomic DNA) or RNA (which includes synthetic RNA) and can be provided in a single stranded (in either the sense or the antisense strand) or a double stranded form. The contemplated nucleic acid molecules can include alterations in the coding regions, non-coding regions, or both. Examples are nucleic acid molecule variants containing alterations which produce silent substitutions, additions, or deletions, but do not alter the properties or activities of the encoded polypeptide, variants or fragments.

In some embodiments, the heterologous nucleic acid molecules which can be introduced into the recombinant host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons to optimize expression levels. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism.

The heterologous nucleic acid molecules can be introduced in the yeast host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

### First Genetic Modification

In accordance with the present disclosure, the recombinant yeast host cell has a first genetic modification to express a heterologous polypeptide. Alternatively or in combination, in accordance with the present disclosure, the recombinant yeast host cell has a first genetic modification to over-express a native polypeptide. In some embodiments, the recombinant yeast host cell has a first genetic modification to both express a heterologous polypeptide and to over-express a native polypeptide. The heterologous polypeptide or the native polypeptide is intended to be secreted/exported and the first genetic modification is also for expressing a signal sequence operatively associated with the heterologous polypeptide and/or the native polypeptide. As such, in the recombinant yeast host cell of the present disclosure, the heterologous or the native polypeptide, when present intracellularly, includes a signal sequence which is cleaved upon secretion/export from the recombinant yeast host cell.

In the context of the present disclosure, when the heterologous polypeptide is expressed or the native polypeptide is overexpressed in a control yeast host cell, it impedes the growth rate of the control yeast host cell (when compared to the growth rate of the parental yeast host cell lacking such first genetic modification). The control yeast host cell expresses the heterologous polypeptide or overexpressed the native polypeptide but does not include the second genetic modification. In an embodiment, the control yeast host cell corresponds to the recombinant yeast host cell but lacks the second genetic modification. This impediment in growth rate can be a reduction of at least 10, 15, 20, 25, 30, 35, 40, 45, 50, 60, 65, 70% or more in the growth rate when compared to the growth rate of the parental yeast host cell and/or the recombinant yeast host cell. In some embodiment, this impediment in growth rate can be a reduction of at least 10% in the growth rate when compared to the growth rate of the parental yeast host cell and/or the recombinant yeast host cell. In some embodiment, this impediment in growth rate can be a reduction of at least 15% in the growth rate when compared to the growth rate of the parental yeast host cell and/or the recombinant yeast host cell. In some embodiment, this impediment in growth rate can be a reduction of at least 20% in the growth rate when compared to the growth rate of the parental yeast host cell and/or the recombinant yeast host cell. In some embodiment, this impediment in growth rate can be a reduction of at least 25% in the growth rate when compared to the growth rate of the parental yeast host cell and/or the recombinant yeast host cell. In some embodiment, this impediment in growth rate can be a reduction of at least 30% in the growth rate when compared to the growth rate of the parental yeast host cell and/or the recombinant yeast host cell. In some embodiment, this impediment in growth rate can be a reduction of at least 35% in the growth rate when compared to the growth rate of the parental yeast host cell and/or the recombinant yeast host cell. In some embodiment, this impediment in growth rate can be a reduction of at least 40% in the growth rate when compared to the growth rate of the parental yeast host cell and/or the recombinant yeast host cell. In some embodiment, this impediment in growth rate can be a reduction of at least 45% in the growth rate when compared to the growth rate of the parental yeast host cell and/or the recombinant yeast host cell. In some embodiment, this impediment in growth rate can be a reduction of at least 50% in the growth rate when compared to the growth rate of the parental yeast host cell and/or the recombinant yeast host cell. In some embodiment, this impediment in growth rate can be a reduction of at least 55% in the growth rate when compared to the growth rate of the parental yeast host cell and/or the recombinant yeast host cell. In some embodiment, this impediment in growth rate can be a reduction of at least 60% in the growth rate when compared to the growth rate of the parental yeast host cell and/or the recombinant yeast host cell. In some embodiment, this impediment in growth rate can be a reduction of at least 65% in the growth rate when compared to the growth rate of the parental yeast host cell and/or the recombinant yeast host cell. In some embodiment, this impediment in growth rate can be a reduction of at least 70% in the growth rate when compared to the growth rate of the parental yeast host cell and/or the recombinant yeast host cell. In a specific embodiment, this reduction in the growth rate of the control yeast host cell is observed when the heterologous polypeptide or the native polypeptide does not include a signal sequence (and are thus expressed intracellularly). In another specific embodiment, this reduction in the growth rate of the control yeast host cell is observed when the heterologous polypeptide or the native polypeptide includes a signal sequence (and are thus secreted/exported).

In some embodiments the heterologous polypeptide or the native polypeptide is an enzyme which can be, without limitation, an hydrolase (E.C. 3.1). An hydrolase includes, without limitation, alpha-acetolactate decarboxylase, aminopeptidase, amylase (including an alpha-amylase), maltogenic alpha-amylase, asparaginase, bromelain, carboxypeptidase, catalase, cellulase, chymosin (including chymosin A and B), cyprosin, ficin, glucoamylase (also known as amyloglucosidase or maltase), glucanase, glucose oxidase, glucose isomerase, hemicellulase, hexose oxidase, inulinase, invertase, lactase, lipase (including a phospholipase), lipoxidase, lysozyme, mannanase, milk coagulating enzyme, pancreatin, papain, pectinase, pentosanase, pepsin, phospholipase, peroxidase, protease, pullulanase, rennet (including bovine rennet), transglutaminase, trypsin, urease, esterase (including a fumonisin esterase) and/or xylanase. In an embodiment, the heterologous polypeptide or native peptide include, without limitation, amylolytic enzymes (including, for example, maltogenic alpha-amylases, glucoamylases, alpha-amylases and fungal amylases), cellulases/hemicellulases, oxidases (including, for example, glucose oxidases), asparaginases, and lipases. In another embodiment, the heterologous polypeptide or the native polypeptide can be, without limitation, a phytase, β-glucanase, xylanase, alpha-galactosidase, protease, amylase, lipase (including a phospholipase), mannanase, cellulase and/or hemicellulasespectinase.

In some embodiments of the present disclosure, the heterologous or native polypeptide is a polypeptide having lipase activity. Lipases (EC 3.1.1.3) are enzymes class of enzymes capable of hydrolyzing lipids. In one embodiment, the heterologous or native polypeptide is a polypeptide having phospholipase activity. Phospholipases (EC 3.1.1.4, EC 3.1.1.5, EC 3.1.1.32, EC 3.1.4.3, and EC 3.1.4.4) are enzymes that hydrolyzes phospholipids into fatty acids and other lipophilic substances. Lipases and phospholipases are part of the family of carboxylic ester (in the case of EC 3.1.1) or phosphoric diester (in the case of EC 3.1.4) hydrolases. In an embodiment, the polypeptide having lipase activity can be a triacylglycerol lipase from Thermomyces lanuginosis, a phospholipase from Fusarium oxysporum (which can have, in some embodiments, the amino acid sequence of SEQ ID NO: 1 or 4, be a variant of the amino acid sequence of SEQ ID NO: 1 or 4, or be a fragment of the amino acid sequence of SEQ ID NO: 1 or 4), a phospholipase A2 from Sus scrofa, a phospholipase A2 from Streptomyces vialaceoruber and/or a phospholipase A2 from Aspergillus oryzea. Embodiments of polypeptides having lipase activity have been described in US20200087672 and WO2018/167670, both of which are incorporated herein in their entirety.

In some embodiments of the present disclosure, the polypeptide having phospholipase activity can have the amino acid sequence of SEQ ID NO: 1 or 4, or be encoded by a nucleic acid sequence (such as a degenerate sequence) encoding the amino acid sequence of SEQ ID NO: 1 or 4. In some embodiments, the polypeptide having phospholipase activity include variants and fragments of the phospholipase polypeptides of SEQ ID NO: 1 or 4 (also referred to herein as phospholipase variants and fragments). The phospholipase variants and fragments have phospholipase activity. Phospholipase activity can be measured by various techniques as known in the art, for example, by using Ped-Al as a substrate as described in the Examples.

In some embodiments of the present disclosure, the heterologous or native polypeptide is a polypeptide having esterase activity. Esterases are able to hydrolyze an ester into an acid and an alcohol. Esterases include, without limitation, acetylesterases (E.C. 3.1.1.6), pectinesterases (E.C. 3.1.1.11), fumonisin (B1) esterases (E.C. 3.1.1.87), thiolester hydrolases (E.C. 3.1.2 which includes thioesterase), phosphoric monoester hydrolases (E.C.3.1.1), phosphoric diester hydrolases (E.C.3.1.4), triphosphoric monoester hydrolases (3.1.5), sulfatases (E.C. 3.1.6), disphosphoric monoester hydrolases (E.C. 3.1.7), phosphoric triester hydrolases (E.C. 3.1.8), exonucleases (E.C. 3.1.11, 3.1.13, 3.1.14, and 3.1.15) as well as endonucleases. In one embodiment, the heterologous or native polypeptide is a polypeptide having fumonisin esterase activity. Fumonisin esterases catalyse the hydrolysis of fumonisin B1 and water into aminopentol as well as propane-1,2,3-tricarboxylate. In an embodiment, the polypeptide having fumonisin esterase activity can be from Sphingopyxis sp., Exophiala sp. (Exophiala spinifera for example) or from Bacterium ATCC 55552. It some embodiments, the polypeptide having fumonisin esterase activity can have the amino acid sequence of SEQ ID NO: 21, be a variant of the amino acid sequence of SEQ ID NO: 21 having fumonisin esterase activity, or be a fragment of the amino acid sequence of SEQ ID NO: 21 having fumonisin esterase activity). Embodiments of polypeptides having fumonisin esterases have been described in U.S. patent No. 6,025,188, herein enclosed in its entirety.

In some embodiments of the present disclosure, the polypeptide having fumonisin esterase activity can have the amino acid sequence of SEQ ID NO: 21, or be encoded by a nucleic acid sequence (including a degenerate sequence) encoding the amino acid sequence of SEQ ID NO: 21. In some embodiments, the polypeptide having fumonisin esterase activity include variants and fragments of the fumonisin esterase polypeptide of SEQ ID NO: 21 (also referred to herein as fumonisin esterase variants and fragments). The fumonisin esterase variants and fragments have fumonisin esterase activity. Fumonisin esterase activity can be determined by those skilled in the art, for example, by measuring tricarballylic acid release from fumonisin (e.g., TCA assay).

In some embodiments of the present disclosure, the heterologous or native polypeptide is a polypeptide having alpha-amylase activity. Alpha-amylases exhibit endohydrolysis activity of (1→4)-alpha-D-glucosidic linkages in polysaccharides containing three or more (1→4)-alpha-linked D-glucose units. Alpha-amylases (E.C. 3.2.1.1) are a subfamily of enzymes of glycosidases (E.C.3.2.1). In an embodiment, the polypeptide having alpha-amylase activity can be from an Archaeal bacterium, such as, for example, from Thermococcus sp. or, in a specific embodiment, from Thermococcus hydrothermalis. It some embodiments, the polypeptide having alpha-amylase activity can have the amino acid sequence of SEQ ID NO: 21, be a variant of the amino acid sequence of SEQ ID NO: 23 having alpha-amylase activity, or be a fragment of the amino acid sequence of SEQ ID NO: 23 having alpha-amylase activity).

In some embodiments of the present disclosure, the polypeptide having alpha-amylase activity can have the amino acid sequence of SEQ ID NO: 23, or be encoded by a nucleic acid sequence (including a degenerate sequence) encoding the amino acid sequence of SEQ ID NO: 23. In some embodiments, the polypeptide having alpha-amylase activity include variants and fragments of the alpha-amylase polypeptide of SEQ ID NO: 23 (also referred to herein as alpha-amylase variants and fragments). The alpha-amylase variants and fragments have alpha-amylase activity. Alpha-amylase activity can be determined easily by those skilled in the art. In some embodiments, the alpha-amylase activity can be determined using the Ceralpha™ assay described in the Examples.

The heterologous or native polypeptide can be a polypeptide having oxidase activity. As used herein, the expression “oxidase” refers to a class of enzymes capable of catalyzing an oxidation-reduction reaction. The oxidase can be an oxidoreductase such as an hexose oxidase (including a glucose oxidase). In one embodiment, the oxidase is a glucose oxidase from Aspergillus niger. Embodiments of polypeptides having oxidase activity have been described in US20200087672 and WO2018/167670, both of which are incorporated herein in their entirety.

As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylases as well as bacterial alpha-amylases, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68), and amylomaltase (EC 2.4.1.25). In an embodiment, the one or more amylolytic enzymes can be an a-amylase from Aspergillus oryzae, a maltogenic α-amylase from Geobacillus stearothermophilus, a glucoamylase from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Embodiments of polypeptides having amylolytic activity have been described in WO 2017/037614, WO 2018/002360, WO 2019/186371, US20200087672 and WO2018/167670, both of which are incorporated herein in their entirety.

As used herein, the expression “cellulase/hemi-cellulase” refers to a class of enzymes capable of hydrolyzing cellulose, hemi-cellulose, or pentosans. Cellulases/hemi-cellulases include, but are not limited to a cellulase (E.C. 3.2.1.4) and an endoB(1,4)D-xylanase (E.C. 3.2.1.8). In an embodiment, the one or more cellulase/hemi-cellulase can be a cellulase from Penicillium funiculosum and/or an endoB(1,4)D-xylanase from Rasamsonia emersonii. Embodiments of polypeptides having cellulase/hemi-cellulase activity have been described in US20200087672 and WO2018/167670, both of which are incorporated herein in their entirety.

As used herein, the expression “asparaginase” refers to a class of enzymes capable of catalyzing the conversion of asparagine into aspartic acid and ammonium. Embodiments of polypeptides having asparaginase activity have been described in US20200087672 and WO2018/167670, both of which are incorporated herein in their entirety.

As used in the present disclosure, the term “maltogenic amylase” refers to a polypeptide capable of hydrolyzing starch or hydrolyzed starch into maltose. Maltogenic amylases include, but are not limited to fungal alpha-amylases (derived, for example, from Aspergillus sp. (e.g., A. Niger, A. kawachi, and A. oryzae); Trichoderma sp. (e.g., T. reesie), Rhisopus sp., Mucor sp., and Penicillium sp.), acid stable fungal amylase (derive, for example, from Aspergillus niger), beta-amylases (derived, for example, from plant (wheat, barley, rye, shorgum, soy, sweet potato, rice) and microorganisms (Bacillus cereus, Bacillus polymixa, Bacillus megaterium, Arabidopsis thaliana), maltogenic amylases (E.C.3.2.1.133) (derived, for example, from microorganisms such as Bacillus subtilis, Geobacillus stearothermophilus, Bacillus thermoalkalophilus, Lactobacillus gasseri, Thermus sp.). In a specific embodiment, the recombinant yeast host cells of the present disclosure include an heterologous nucleic acid molecule coding for the heterologous maltogenic amylase derived from Geobacillus stearothermophilus. Embodiments of polypeptides having maltogenic amylase activity have been described in US20200087672 and WO2018/167670, both of which are incorporated herein in their entirety.

As used herein, the expression “phosphatase” refers to a polypeptide capable, in the presence of water, of catalyzing the cleavage of a phosphoric acid monoester into a phosphate ion and an alcohol. An embodiment of a phosphatase is a phytase, a protein having enzymatic activity and capable of catalyzing the hydrolysis of phytic acid (myo-inositol hexakisphosphate) into inorganic phosphorus. There are four distinct classes of phytase: histidine acid phosphatases (HAPS), beta-propeller phytases, purple acid phosphatases and protein tyrosine phosphatase-like phytases (PTP-like phytases). Phytic acid has six phosphate groups that may be released by phytases at different rates and in different order. Phytases hydrolyze phosphates from phytic acid in a stepwise manner, yielding products that again become substrates for further hydrolysis. Phytases have been grouped based on the first phosphate position of phytic acid that is hydrolyzed: are 3-phytase (EC 3.1.3.8), 4-phytase (EC 3.1.3.26) and 5-phytase (EC 3.1.3.72). In an embodiment, the phytase is derived from a bacterial species, such as, for example, a Citrobacter sp. or an Escherichia sp. In a specific embodiment, the heterologous phytase is derived from a Citrobacter sp., such as for example Citrobacter braakii. In another embodiment, the heterologous phytase is derived from an Escherichia sp., such as, for example, Escherichia coli. Embodiments of polypeptides having phosphatase activity have been described in US20200087672 and WO2018/167670, both of which are incorporated herein in their entirety.

A variant comprises at least one amino acid difference (substitution or addition) when compared to the amino acid sequence of the heterologous or native polypeptide. The variants do exhibit one or more biological activity of the heterologous or native polypeptide. In an embodiment, the variant polypeptide exhibits at least 50%, 60%, 70%, 80%, 90%, 95%, 96%, 97%, 98% or 99% of the biological activity of the wild-type polypeptide. The variants also have at least 70%, 80%, 85%, 90%, 95%, 96%, 97%, 98% or 99% identity to the amino acid sequence of the wild-type polypeptide. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLB 1, GAP PENALTY=3, WINDOW=5 and DIAGONALS SAVED=5.

The variant described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant can also be a conservative variant or an allelic variant. As used herein, a conservative variant refers to alterations in the amino acid sequence that do not adversely affect the biological functions of the polypeptide. A substitution, insertion or deletion is said to adversely affect the protein when the altered sequence prevents or disrupts a biological function associated with the wild-type polypeptide. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the phospholipase.

In some embodiments of the present disclosure, the polypeptides can be fragments of the heterologous or native polypeptide or fragments from the variants described herein. A fragment comprises at least one less amino acid residue when compared to the amino acid sequence of the wild-type polypeptide or variant and still possess the biological activity of the full-length polypeptide. In an embodiment, the fragment exhibits at least 50%, 60%, 70%, 80%, 90%, 95%, 96%, 97%, 98% or 99% of the biological activity of full-length polypeptide or variant thereof. The fragments can also have at least 70%, 80%, 85%, 90%, 95%, 96%, 97%, 98% or 99% identity to the amino acid sequence the polypeptide or a variant thereof over its entire length. The fragment can be, for example, a truncation of one or more amino acid residues at the amino-terminus, the carboxy terminus or both termini of the phospholipase polypeptide or variant. Alternatively or in combination, the fragment can be generated from removing one or more internal amino acid residues. In an embodiment, the fragment has at least 100, 150, 200, 250, 300, 350, 400, 450, 500 or more consecutive amino acids of the polypeptide or the variant.

In accordance with the present disclosure, the heterologous polypeptide expressed in the recombinant yeast host cell or native polypeptide overexpressed in the recombinant yeast host cell is intended to be secreted/exported. As such, it is associated intracellularly with a signal sequence. As used herein, polypeptides “associated with a signal sequence” refers to polypeptides which are transcribed and translated with a signal sequence, and the signal sequence is subsequently cleaved as the polypeptide is processed in the secretion pathway. In some embodiments, the heterologous nucleic acid molecules include a coding sequence for one or a combination of signal sequence(s) allowing the export of the heterologous polypeptide or the native polypeptide outside the yeast host cell's wall. The signal sequence can simply be added to the heterologous nucleic acid molecule (usually in frame with the sequence encoding the heterologous or native polypeptide) or replace the signal sequence already present in the heterologous or native polypeptide. The signal sequence can be native or heterologous to the nucleic acid sequence encoding the heterologous or native polypeptide. In some embodiments, one or more signal sequences can be used.

In one embodiment, the signal sequence is an heterologous signal sequence, such as, for example, the heterologous signal sequence is from an invertase protein, an AGA2 protein or a fungal amylase. In an embodiment, the heterologous signal peptide is from the invertase protein. In still another embodiment, the heterologous signal sequence is from the AGA2 protein. In still another embodiment, the heterologous signal sequence is from the fungal amylase. In some embodiments, the signal sequence can be from the SUC2 gene and have, for example, the amino acid sequence MLLQAFLFLLAGFAAKISA (SEQ ID NO: 5), be a variant thereof or be a fragment thereof. In some embodiments, the signal sequence can be from the PDI1 gene and have, for example, the amino acid sequence MKFSAGAVLSWSSLLLASSVFAQQEAVA (SEQ ID NO: 6), be a variant thereof or be a fragment thereof. In some embodiments, the signal sequence can be from the gene encoding the M1 killer protein and have, for example, the amino acid sequence MTKPTQVLVRSVSILFFITLLHLVVA (SEQ ID NO: 7), be a variant thereof or be a fragment thereof. In some embodiments, the signal sequence can be from the gene encoding the alpha-mating factor and have, for example, the amino acid sequence MRFPSIFTAVLFAASSALA (SEQ ID NO: 8), be a variant thereof or be a fragment thereof. In some embodiments, the signal sequence can be an hybrid from the OST1 gene and the alpha-mating factor signal sequence and have, for example, the amino acid sequence MRQVWFSWIVGLFLCFFNVSSAAPVNTTTEDETAQIPAEAVIGYSDLEGDFDVAVLPFSNSTNN GLLFINTTIASIAAKEEGVSLEKREAEA (SEQ ID NO: 2), be a variant thereof or be a fragment thereof. In some embodiments, the signal sequence can be from the a phospholipase gene and have, for example, the amino acid sequence MLLLPLLSAITLAVA (SEQ ID NO: 3), be a variant thereof or be a fragment thereof. In some embodiments, the signal sequence can be from the a phospholipase gene and have, for example, the amino acid sequence MLLLPLLSAITLAVASPVALDDYVNSLEER (SEQ ID NO: 9), be a variant thereof or be a fragment thereof. In still another embodiment, the signal sequence can be from an alpha-mating factor gene and can have, for example, the amino acid sequence MRFPSIFTAVLFAASSALA (SEQ ID NO: 23), be a variant thereof or be a fragment thereof. Embodiments of signal sequences have been described in US20200087672 and WO2018/167670, both of which are incorporated herein in their entirety.

In preferred embodiments, the heterologous or native polypeptide is expressed to be secreted out of the cell to the extracellular space. The heterologous or native polypeptide is transported along the secretory pathway of the recombinant yeast host cell and secreted into the extracellular space or the culture medium in a free form (not associated with a surface of the host cell).

In other embodiments, the heterologous or native polypeptide is “cell-associated” to the recombinant yeast host cell because it is designed to be expressed, exported and remain physically associated with the recombinant yeast host cells. In one embodiment, the heterologous or native polypeptide can be secreted, but if it is, it must remain physically associated with the recombinant yeast host cell. In one embodiment, at least one portion (usually at least one terminus) of the heterologous or native polypeptide is bound, covalently, non-covalently and/or electrostatically for example, to the cell wall (and in some embodiments to the cytoplasmic membrane). For example, the heterologous or native polypeptide can be modified to bear one or more transmembrane domains, to have one or more lipid modifications (myristoylation, palmitoylation, farnesylation and/or prenylation), to interact with one or more membrane-associated protein and/or to interactions with the cellular lipid rafts. While the heterologous or native polypeptide may not be directly bound to the cell membrane or cell wall (e.g., such as when binding occurs via a tethering moiety), the protein is nonetheless considered a “cell-associated” heterologous polypeptide or native polypeptide according to the present disclosure.

In some embodiments, the heterologous or native polypeptide can be expressed to be located at and associated to the cell wall or the cell membrane of the recombinant yeast host cell. In some embodiments, the heterologous polypeptide or native polypeptide is expressed, exported, and transported to be located at and associated to the external surface of the cell wall or cell membrane of the host cell. Recombinant yeast host cells all have a cell wall (which includes a cytoplasmic membrane) defining the intracellular (e.g., internally-facing the nucleus) and extracellular (e.g., externally-facing) environments. The heterologous or native polypeptide can be located at (and in some embodiments, physically associated to) the external face of the recombinant yeast host's cell wall and, in further embodiments, to the external face of the recombinant yeast host's cytoplasmic membrane. In the context of the present disclosure, the expression “associated to the external face of the cell wall/cytoplasmic membrane of the recombinant yeast host cell” refers to the ability of the heterologous or native polypeptide to physically integrate (in a covalent or non-covalent fashion), at least in part, in the cell wall (and in some embodiments in the cytoplasmic membrane) of the recombinant yeast host cell. The physical integration can be attributed to the presence of, for example, a transmembrane domain on the heterologous or native polypeptide, a domain capable of interacting with a cytoplasmic membrane protein on the heterologous polypeptide or native polypeptide, a post-translational modification made to the heterologous enzyme or native polypeptide (e.g., lipidation), etc.

Some heterologous or native polypeptide have the intrinsic ability to locate at and associate to the cell wall of a recombinant yeast host cell (e.g., being cell-associated).

In some embodiments, the heterologous polypeptide is provided as a chimeric construct by combining it with a tethering amino acid moiety which will provide or increase attachment to the cell wall of the recombinant yeast host cell. In such embodiment, the chimeric or native polypeptide will be considered “tethered”. It is preferred that the amino acid tethering moiety of the chimeric protein be neutral with respect to the biological (enzymatic) activity of the heterologous or native polypeptide, e.g., does not interfere with the biological (enzymatic) activity of the heterologous or native polypeptide. In some embodiments, the association of the amino acid tethering moiety with the heterologous or native polypeptide can increase the biological (enzymatic) activity and/or stability of the heterologous or native polypeptide (when compared to the non-tethered, non-chimeric form).

In an embodiment, a tethering moiety can be used to be expressed with the heterologous or native polypeptide to locate the polypeptide to the wall of the recombinant yeast host cell. Various tethering amino acid moieties are known art and can be used in the chimeric proteins of the present disclosure.

The tethering moiety can be a transmembrane domain found on another protein and allow the chimeric protein to have a transmembrane domain. In such embodiment, the tethering moiety can be derived from the FLO1 protein. Embodiments of tethering moieties and their variants and fragments have been described in US20200087672 and WO2018/167670, both of which are incorporated herein in their entirety.

In still another example, the amino acid tethering moiety can be modified post-translation to include a glycosylphosphatidylinositol (GPI) anchor and allow the chimeric protein to have a GPI anchor. GPI anchors are glycolipids attached to the terminus of a protein (and in some embodiments, to the carboxyl terminus of a protein) which allows the anchoring of the protein to the cytoplasmic membrane of the cell membrane. Tethering amino acid moieties capable of providing a GPI anchor include, but are not limited to those associated with/derived from a SED1 protein, a TIR1 protein, a CWP2 protein, a CCW12 protein, a SPI1 protein, a PST1 protein or a combination of a AGA1 and a AGA2 protein. In an embodiment, the tethering moiety provides a GPI anchor and, in still a further embodiment, the tethering moiety is derived from the SPI1 protein or the CCW12 protein. In embodiments in which the tethering moiety is derived from the CCW12 protein, it can have, for example, the amino acid sequence of SEQ ID NO: 26, be a variant thereof or a fragment thereof. Embodiments of tethering amino acid moieties capable of providing a GPI anchor have been described in US20200087672 and WO2018/167670, both of which are incorporated herein in their entirety.

The tethering amino acid moiety can be a variant of a known/native tethering amino acid moiety. A variant comprises at least one amino acid difference when compared to the amino acid sequence of the native tethering amino acid moiety. As used herein, a variant refers to alterations in the amino acid sequence that do not adversely affect the biological functions of the tethering amino acid moiety (e.g., the location on the external face and the anchorage of the heterologous polypeptide or native polypeptide in the cytoplasmic membrane). A substitution, insertion or deletion is said to adversely affect the protein when the altered sequence prevents or disrupts a biological function associated with the tethering amino acid moiety (e.g., the location on the external face and the anchorage of the heterologous polypeptide or native polypeptide in the cytoplasmic membrane). For example, the overall charge, structure or hydrophobic-hydrophilic properties of the protein can be altered without adversely affecting a biological activity. Accordingly, the amino acid sequence can be altered, for example to render the tethering moiety more hydrophobic or hydrophilic, without adversely affecting the biological activities of the tethering amino acid moiety. The tethering amino acid moiety variants have at least 50%, 55%, 60%, 65%, 70%, 75%, 80%, 85%, 90%, 95%, 96%, 97%, 98% or 99% identity to the tethering amino acid moieties described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant tethering amino acid moieties described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” of the tethering amino acid moiety can be a conservative variant or an allelic variant.

The tethering amino acid moiety can be a fragment of a known/native tethering amino acid moiety or fragment of a variant of a known/native tethering amino acid moiety. Tethering amino acid moiety “fragments” have at least at least 10, 20, 30, 40, 50, 60, 70, 80, 90, 100 or more consecutive amino acids of the tethering amino acid moiety. A fragment comprises at least one less amino acid residue when compared to the amino acid sequence of the known/native tethering amino acid moiety and still possess the biological activity of the full-length tethering amino acid moiety (e.g., the location to the cell wall).

In embodiments in which an amino acid tethering moiety is desirable, the heterologous or native polypeptide can be provided as a chimeric protein expressed by the recombinant yeast host cell and having one of the following formulae (provided from the amino (NH₂) to the carboxyl (COOH) orientation):

(N₂) SS-TT-L-PP (COOH) (I)

(NH₂) SS-PP-L-TT (COOH) (II)

wherein:

PP is the heterologous or native polypeptide;

L is present or absent and is an amino acid linker;

TT is present or absent and is an amino acid tethering moiety for associating the polypeptide to a cell wall or cell membrane of the recombinant yeast host cell;

SS is a signal sequence moiety;

(NH₂) indicates the amino terminus of the heterologous polypeptide;

(COOH) indicates the carboxyl terminus of the heterologous polypeptide; and

“-” is an amide linkage.

When the amino acid linker (L) is absent, the tethering amino acid moiety is directly associated with the heterologous polypeptide. In the chimeras of formulae (I), this means that the carboxyl terminus of the signal sequence moiety is directed associated to the amino terminus of the tethering amino acid moiety, and the carboxyl terminus of the tethering amino acid moiety is associated (directly in the absence of the linker or indirectly in the presence of the linker) to the amino terminus of the heterologous polypeptide. In some embodiments, this means that the carboxyl terminus of the signal sequence moiety is directed associated to the amino terminus of the tethering moiety, and the carboxyl terminus of tethering moiety is directly associated to the amino terminus of the heterologous/native polypeptide. In the chimeras of formulae (II), this means that the carboxyl terminus of the signal sequence moiety is directed associated to the amino terminus of the heterologous/native polypeptide, and the carboxyl terminus of the heterologous/native polypeptide is associated (directly in the absence of the linker and indirectly in the presence of the linker) to the amino terminus of the tethering moiety. In some embodiments, this means that the carboxyl terminus of the signal sequence moiety is directed associated to the amino terminus of the heterologous/native polypeptide, and the carboxyl terminus of heterologous/native polypeptide is directly associated to the amino terminus of the tethering amino acid moiety.

In some embodiments, the presence of an amino acid linker (L) is desirable for example: to provide some flexibility or a desired level of flexibility between the heterologous polypeptide moiety and the tethering amino acid moiety; to provide rigidity between the heterologous polypeptide moiety and the tethering amino acid moiety; to facilitate the construction of the heterologous nucleic acid molecule; to provide better solubility of the heterologous polypeptide moiety; to provide higher expression of the heterologous polypeptide moiety; to facilitate folding of the polypeptide moiety; to improve the biological activity of the heterologous polypeptide moiety; and/or to introduce a cleavage site for release of the heterologous polypeptide moiety from the cell. As used in the present disclosure, the “amino acid linker” or “L” refer to a stretch of one or more amino acids separating the heterologous polypeptide PP and the amino acid tethering moiety TT (e.g., indirectly linking the heterologous polypeptide or native polypeptide to the amino acid tethering moiety TT). It is preferred that the amino acid linker be neutral, e.g., does not interfere with the biological (enzymatic) activity of the heterologous polypeptide or native polypeptide, nor with the biological (cell-association) activity of the amino acid tethering moiety. In some embodiments, the amino acid linker L can increase the biological activity of the heterologous polypeptide or native polypeptide moiety and/or of the amino acid tethering moiety.

Various amino acid linkers exist and include, without limitations, (G)ₙ, (GS)ₙ; (GGS)ₙ; (GGGS)ₙ; (GGGGS)ₙ; (GGSG)ₙ; (GSAT)ₙ, wherein n=is an integer between 1 to 8 (or more). In an embodiment, the amino acid linker L is (GGGGS)ₙ (also referred to as G₄S) and, in still further embodiments, the amino acid linker L comprises more than one G₄S motifs (SEQ ID NO: 10). For example, the amino acid linker L can be (G₄S)₃ and have the amino acid sequence of SEQ ID NO: 11. In another example, the amino acid linker L can be (G)₈ and have the amino acid sequence of SEQ ID NO: 12. In still another example, the amino acid linker L can be (G₄S)₈ and have the amino acid sequence of SEQ ID NO: 13. The amino acid linker can also be, in some embodiments, GSAGSAAGSGEF (SEQ ID NO: 14).

Additional amino acid linkers exist and include, without limitations, (EAAK)ₙ and (EAAAK)ₙ, wherein n=is an integer between 1 to 8 (or more). In some embodiments, the one or more (EAAK)ₙ/(EAAAK)ₙ motifs can be separated by one or more additional amino acid residues. In an embodiment, the amino acid linker comprises one or more EA₂K (SEQ ID NO: 15) or EA₃K (SEQ ID NO: 16) motifs. In an embodiment, the amino acid linker can be (EAAK)₃ and has the amino acid sequence of SEQ ID NO: 17. In another embodiment, the amino acid linker can be (A(EAAAK)₄ ALEA(EAAAK)₄A) and has the amino acid sequence of SEQ ID NO: 18. In yet another embodiment, the amino acid linker can have the amino acid sequence of SEQ ID NO: 25.

Further amino acid linkers include those having one or more (AP), motifs wherein n=is an integer between 1 to 10 (or more). In an embodiment, the linker is (AP) 10 and has the amino acid of SEQ ID NO: 19. In some embodiments, the linker also includes one or more HA tag (SEQ ID NO: 20). Embodiments of amino acid linkers have been described in US20200087672 and WO2018/167670, both of which are incorporated herein in their entirety.

### Second Genetic Modification

The recombinant yeast host cell of the present disclosure also includes a second genetic modification. The second genetic modification is aimed at increasing the growth rate of the recombinant yeast expressing the heterologous polypeptide or overexpressing the native polypeptide (when compared for example with the control yeast host cell). The presence of the second genetic modification restores, at least in part, the growth rate of the recombinant yeast host cell when compared to the parental yeast host cell. By introducing the second genetic modification, the recombinant yeast host cell exhibits an increase in growth rate of at least 100, 105, 110, 115, 120, 125, 130, 135, 140, 145, 150, 155, 160, 165, 170, 175, 180, 185, 190, 195, 200, 205, 210, 215, 200% or more when compared to the growth rate of the control yeast host cell (determined under similar conditions). In an embodiment, the recombinant yeast host cell exhibits an increase in growth rate of at least 100% or more when compared to the growth rate of the control yeast host cell. In an embodiment, the recombinant yeast host cell exhibits an increase in growth rate of at least 105% or more when compared to the growth rate of the control yeast host cell. In an embodiment, the recombinant yeast host cell exhibits an increase in growth rate of at least 110% or more when compared to the growth rate of the control yeast host cell. In an embodiment, the recombinant yeast host cell exhibits an increase in growth rate of at least 115% or more when compared to the growth rate of the control yeast host cell. In an embodiment, the recombinant yeast host cell exhibits an increase in growth rate of at least 120% or more when compared to the growth rate of the control yeast host cell. In an embodiment, the recombinant yeast host cell exhibits an increase in growth rate of at least 125% or more when compared to the growth rate of the control yeast host cell. In an embodiment, the recombinant yeast host cell exhibits an increase in growth rate of at least 130% or more when compared to the growth rate of the control yeast host cell. In an embodiment, the recombinant yeast host cell exhibits an increase in growth rate of at least 135% or more when compared to the growth rate of the control yeast host cell. In an embodiment, the recombinant yeast host cell exhibits an increase in growth rate of at least 140% or more when compared to the growth rate of the control yeast host cell. In an embodiment, the recombinant yeast host cell exhibits an increase in growth rate of at least 145% or more when compared to the growth rate of the control yeast host cell. In an embodiment, the recombinant yeast host cell exhibits an increase in growth rate of at least 150% or more when compared to the growth rate of the control yeast host cell. In an embodiment, the recombinant yeast host cell exhibits an increase in growth rate of at least 155% or more when compared to the growth rate of the control yeast host cell. In an embodiment, the recombinant yeast host cell exhibits an increase in growth rate of at least 160% or more when compared to the growth rate of the control yeast host cell. In an embodiment, the recombinant yeast host cell exhibits an increase in growth rate of at least 165% or more when compared to the growth rate of the control yeast host cell. In an embodiment, the recombinant yeast host cell exhibits an increase in growth rate of at least 170% or more when compared to the growth rate of the control yeast host cell. In an embodiment, the recombinant yeast host cell exhibits an increase in growth rate of at least 175% or more when compared to the growth rate of the control yeast host cell. In an embodiment, the recombinant yeast host cell exhibits an increase in growth rate of at least 180% or more when compared to the growth rate of the control yeast host cell. In an embodiment, the recombinant yeast host cell exhibits an increase in growth rate of at least 185% or more when compared to the growth rate of the control yeast host cell. In an embodiment, the recombinant yeast host cell exhibits an increase in growth rate of at least 190% or more when compared to the growth rate of the control yeast host cell. In an embodiment, the recombinant yeast host cell exhibits an increase in growth rate of at least 195% or more when compared to the growth rate of the control yeast host cell. In an embodiment, the recombinant yeast host cell exhibits an increase in growth rate of at least 200% or more when compared to the growth rate of the control yeast host cell. In an embodiment, the recombinant yeast host cell exhibits an increase in growth rate of at least 205% or more when compared to the growth rate of the control yeast host cell. In an embodiment, the recombinant yeast host cell exhibits an increase in growth rate of at least 210% or more when compared to the growth rate of the control yeast host cell. In an embodiment, the recombinant yeast host cell exhibits an increase in growth rate of at least 215% or more when compared to the growth rate of the control yeast host cell. In an embodiment, the recombinant yeast host cell exhibits an increase in growth rate of at least 220% or more when compared to the growth rate of the control yeast host cell. In some embodiments, the recombinant yeast host cell exhibits a restoration of growth rate to a similar level as the parental yeast host cell's growth rate. In other embodiments, the recombinant yeast host cell exhibits a partial restoration of growth rate relative to the parental yeast host cell growth rate. This increase in growth rate is observed when one compares the growth rate of the control yeast host cell which expresses the heterologous polypeptide or the native polypeptide (in the presence or in the absence of the signal sequence) but lacks the second genetic modification. In one embodiment, this increase in growth rate is observed when one compares the growth rate of a control yeast host cell which expresses the first genetic modification together with a signal sequence (e.g., the first genetic modification).

In some embodiments, the second genetic modification improves or enhances the secretion and/or the trafficking of the heterologous polypeptide or the native polypeptide, thereby limiting the impact of the expression of the heterologous polypeptide or the native polypeptide on the growth rate of the recombinant yeast host cell. The recombinant yeast host cell of the present disclosure can include one or more second genetic modifications. The second genetic modification modulates the expression of one or more specific gene (which may be native or heterologous). The second genetic modification can cause the increase in the expression (e.g., overexpression) of a native or an heterologous gene. The second modification can cause the decrease in the expression (and in some embodiments the inactivation) or a native gene.

In some embodiments, the second genetic modification favors the secretion of the heterologous polypeptide or the overexpressed native polypeptide. The secretion of a protein involves numerous steps from translation to post-translational modifications to trafficking, etc. In some embodiments, the second genetic modification can include the modification in one or more of the following pathways: the translation pathway, the post-translational modification pathway, the protein trafficking pathway, the vacuolar pathway or the cell wall stability pathway. In some embodiments, the second genetic modification can include the protein secretory and trafficking pathway. As used in the context of the present disclosure, the “translation pathway” refers to genes and proteins involved in the translation of a messenger RNA (mRNA) molecule into a protein. A genetic modification in the translation pathway which would favor the translation of a mRNA molecule into a protein would be considered as a second genetic modification of the present disclosure. A genetic modification in the translation pathway which would limit the biological activity of an inhibitor of the translation of a mRNA molecule into a protein would be considered as a second genetic modification of the present disclosure. The “post-translational pathway” refers to genes and proteins involved in the modification of the secondary, tertiary or quaternary structure of a protein. Such modifications include, without limitation, the folding and/or the glycosylation of newly translated proteins. The events of the post-translational pathway usually occur in the endoplasmic reticulum (ER) and as such the second genetic modification can target a protein which is expressed in the ER. A genetic modification which would favor the appropriate modification of the newly translated protein would be considered as a second genetic modification of the present disclosure. A genetic modification which would limit the biological activity of an inhibitor of a post-translational modification of the newly translated protein would be considered as a second genetic modification of the present disclosure. The “protein trafficking pathway” refers to genes and proteins involved in the trafficking of the proteins between the ER, the Golgi system and the membrane. A genetic modification which would favor the trafficking of a protein from the ER to the Golgi system or from the Golgi system to the membrane would be considered as a second genetic modification of the present disclosure. A genetic modification which would limit the retrograde trafficking of a protein from the membrane to the Golgi system or from the Golgi system to the ER would be considered to be a second genetic modification according to the present disclosure. The “vacuolar pathway” refers to genes and proteins involved in the formation of vacuoles and the vacuolar degradations. A genetic modification which would limit the formation of vacuoles and/or the vacuolar protein degradation would be considered a second genetic modification of the present disclosure. A genetic modification which would favor the biological activity of an inhibitor of the formation of vacuole and/or the vacuolar protein degradation would be considered a second genetic modification of the present disclosure. The “cell wall stability pathway” refers to genes and proteins involved in the maintenance of the integrity of the cell wall. A genetic modification which would favor the reduction in the stability of the cell wall would be considered a second genetic modification according to the present disclosure.

The second genetic modification can include introducing one or more copies of a heterologous gene encoding a heterologous polypeptide in the recombinant yeast host cell. Alternatively or in combination, the second genetic modification can include introducing a different promoter (which in some embodiments can include replacing the native promoter with a heterologous promoter) for controlling the expressing of one or more copies of a native gene, a gene ortholog or a gene paralog. Alternatively or in combination, the second genetic modification can include introducing a different terminator (which in some embodiments can include replacing the native terminator with a heterologous terminator) for controlling the expressing of one or more copies of a native gene, a gene ortholog or a gene paralog. Alternatively or in combination, the second genetic modification can include inactivating at least one copy or all copies of a native gene, a gene ortholog or a gene paralog. The inactivation (partial or complete) of the native gene (including orthologs and paralogs thereof) can be done, for example, by removing at least one nucleotide residue in the native gene. The second genetic modification can be done in one or more locus where the native gene is present. In an embodiment, the second genetic modification can be done in all loci where the native gene is present. The second genetic modification can also be done, for example, by modifying one or more regulatory region present in the native gene. In an embodiment, the second genetic modification can also be done by inserting one or more copies of an heterologous gene (which can be, in some embodiments, one or more additional copies of a native gene of the recombinant yeast host cell), along with the native and/or heterologous regulatory elements (promoter(s) and/or terminator(s)), into any locus of the chromosome.

In an embodiment, the recombinant yeast host cell comprises one or more second genetic modification in the translational pathway. The second genetic modification can be located in a gene encoding a ribosomal protein. The second genetic modification can cause the modulation in the expression (increased or decreased expression) of a native gene encoding a ribosomal protein and/or can cause the expression of a heterologous gene encoding a ribosomal protein. In a specific embodiment, the second genetic modification can cause the modulation in the expression of a native BFR2 gene (and, in some embodiments bearing the Saccharomyces genome database ID SGD:S000002707, a variant thereof or a fragment thereof), a BFR2 gene ortholog or a BFR2 gene paralog and/or the expression of an heterologous polypeptide encoded by a BFR2 gene (and, in some embodiments bearing the Saccharomyces genome database ID SGD:S000002707, a variant thereof or a fragment thereof), a BFR2 gene ortholog or a BFR2 gene paralog. In another specific embodiment, the second genetic modification can cause the modulation in the expression of a native RPP0 gene (and, in some embodiments bearing the Saccharomyces genome database IDSGD:S000004332, a variant thereof or a fragment thereof), a RPP0 gene ortholog or a RPP0 gene paralog and/or the expression of an heterologous polypeptide encoded by a RPP0 gene (and, in some embodiments bearing the Saccharomyces genome database IDSGD:S000004332, a variant thereof or a fragment thereof), a RPP0 gene ortholog or a RPP0 gene paralog. The second genetic modification can cause the modulation in the expression of a native gene encoding a subunit of a signal recognition particle and/or can cause the expression of a heterologous gene encoding a subunit of a signal recognition particle. In a specific embodiment, the second genetic modification can cause the modulation in the expression of a native SRP14 gene (and, in some embodiments bearing the Saccharomyces genome database ID SGD:S000002250, a variant thereof or a fragment thereof), a SRP14 gene ortholog or a SRP14 gene paralog and/or the expression of an heterologous polypeptide encoded by a SRP14 gene (and, in some embodiments bearing the Saccharomyces genome database ID SGD:S000002250, a variant thereof or a fragment thereof), a SRP14 gene ortholog or a SRP14 gene paralog. In a specific embodiment, the second genetic modification can cause the modulation in the expression of a native SRP54 gene (and, in some embodiments bearing the Saccharomyces genome database ID SGD:S000006292, a variant thereof or a fragment thereof), a SRP54 gene ortholog or a SRP54 gene paralog and/or the expression of an heterologous polypeptide encoded by a SRP54 gene (and, in some embodiments bearing the Saccharomyces genome database ID SGD:S000006292, a variant thereof or a fragment thereof), a SRP54 gene ortholog or a SRP54 gene paralog.

The second genetic modification can be located in a gene encoding a protein involved in protein folding. The second genetic modification can cause the modulation in the expression (increasing or decreasing the expression) of a native gene encoding a protein involved in protein folding (such as for example, a component of a chaperone complex such as an ATPase, a molecular chaperone or a nuclear exchange factor) and/or can cause the expression of a heterologous gene encoding a protein involved in protein folding (such as, for example, a component of a chaperone complex such as an ATPase, a molecular chaperone or a nuclear exchange factor). In some embodiments, the second genetic modification can cause the increase of the expression of a native gene encoding a protein involved in protein folding (such as for example, a component of a chaperone complex such as an ATPase, a molecular chaperone or a nuclear exchange factor) and/or can cause the increase in the expression of a heterologous gene encoding a protein involved in protein folding (such as, for example, a component of a chaperone complex such as an ATPase, a molecular chaperone or a nuclear exchange factor). In a specific embodiment, the second genetic modification can cause the modulation in the expression (e.g., in some embodiments, the increase in the expression) of a native KAR2 gene (and, in some embodiments bearing the Saccharomyces genome database SGD:S000003571, a variant thereof or a fragment thereof), a KAR2 gene ortholog or a KAR2 gene paralog and/or the expression of an heterologous polypeptide encoded by a KAR2 gene (and, in some embodiments bearing the Saccharomyces genome database ID SGD:S000003571, a variant thereof or a fragment thereof), a KAR2 gene ortholog or a KAR2 gene paralog. In a specific embodiment, the second genetic modification can cause the modulation in the expression of a native LHS1 gene (and, in some embodiments bearing the Saccharomyces genome database SGD:S000001556, a variant thereof or a fragment thereof), a LHS1 gene ortholog or a LHS1 gene paralog and/or the expression of an heterologous polypeptide encoded by a LHS1 gene (and, in some embodiments bearing the Saccharomyces genome database ID SGD:S000001556, a variant thereof or a fragment thereof), a LHS1 gene ortholog or a LHS1 gene paralog. In a specific embodiment, the second genetic modification can cause the modulation in the expression of a native JEM1 gene (and, in some embodiments bearing the Saccharomyces genome database SGD:S000003609, a variant thereof or a fragment thereof), a JEM1 gene ortholog or a JEM1 gene paralog and/or the expression of an heterologous polypeptide encoded by a JEM1 gene (and, in some embodiments bearing the Saccharomyces genome database ID SGD:S000003609, a variant thereof or a fragment thereof), a JEM1 gene ortholog or a JEM1 gene paralog. In a specific embodiment, the second genetic modification can cause the modulation in the expression of a native SSA1 gene (and, in some embodiments bearing the Saccharomyces genome database SGD:S000000004, a variant thereof or a fragment thereof), a SSA1 gene ortholog or a SSA1 gene paralog and/or the expression of an heterologous polypeptide encoded by a SSA1 gene (and, in some embodiments bearing the Saccharomyces genome database ID SGD:S000000004, a variant thereof or a fragment thereof), a SSA1 gene ortholog or a SSA1 gene paralog. In a specific embodiment, the second genetic modification can cause the modulation in the expression of a native SSE1 gene (and, in some embodiments bearing the Saccharomyces genome database SGD:S000006027, a variant thereof or a fragment thereof), a SSE1 gene ortholog or a SSE1 gene paralog and/or the expression of an heterologous polypeptide encoded by a SSE1 gene (and, in some embodiments bearing the Saccharomyces genome database ID SGD:S000006027, a variant thereof or a fragment thereof), a SSE1 gene ortholog or a SSE1 gene paralog. In a specific embodiment, the second genetic modification can cause the modulation in the expression of a native SIL1 gene (and, in some embodiments bearing the Saccharomyces genome database SGD:S000005391, a variant thereof or a fragment thereof), a SIL1 gene ortholog or a SIL1 gene paralog and/or the expression of an heterologous polypeptide encoded by a SIL1 gene (and, in some embodiments bearing the Saccharomyces genome database ID SGD:S000005391, a variant thereof or a fragment thereof), a SIL1 gene ortholog or a SIL1 gene paralog. In a specific embodiment, the second genetic modification can cause the modulation in the expression of a native SSA4 gene (and, in some embodiments bearing the Saccharomyces genome database SGD:S000000905, a variant thereof or a fragment thereof), a SSA4 gene ortholog or a SSA4 gene paralog and/or the expression of an heterologous polypeptide encoded by a SSA4 gene (and, in some embodiments bearing the Saccharomyces genome database ID SGD:S000000905, a variant thereof or a fragment thereof), a SSA4 gene ortholog or a SSA4 gene paralog. In a specific embodiment, the second genetic modification can cause the modulation in the expression of a native HSF1 gene (and, in some embodiments bearing the Saccharomyces genome database SGD:S000003041, a variant thereof or a fragment thereof), a HSF1 gene ortholog or a HSF1 gene paralog and/or the expression of an heterologous polypeptide encoded by a HSF1 gene (and, in some embodiments bearing the Saccharomyces genome database ID SGD:S000003041, a variant thereof or a fragment thereof), a HSF1 gene ortholog or a HSF1 gene paralog.

The second genetic modification can be located in a gene encoding a protein involved in the unfolded protein response (UPR). The second genetic modification can cause the modulation in the expression (increasing or decreasing the expression) of a native gene encoding a transcription factor favoring the UPR or a component of the UPR and/or can cause the expression of a heterologous gene encoding a protein favoring the UPR or a component of the UPR. In a specific embodiment, the second genetic modification can cause the modulation in the expression of a native HAC1 gene (and, in some embodiments bearing the Saccharomyces genome database SGD:S000001863, a variant thereof or a fragment thereof), a HAC1 gene ortholog or a HAC1 gene paralog and/or the expression of an heterologous polypeptide encoded by a HAC1 gene (and, in some embodiments bearing the Saccharomyces genome database ID SGD:S000001863, a variant thereof or a fragment thereof), a HAC1 gene ortholog or a HAC1 gene paralog. In a specific embodiment, the second genetic modification can cause the modulation in the expression of a native IRE1 gene (and, in some embodiments bearing the Saccharomyces genome database SGD:S000001121, a variant thereof or a fragment thereof), a IRE1 gene ortholog or a IRE1 gene paralog and/or the expression of an heterologous polypeptide encoded by a IRE1 gene (and, in some embodiments bearing the Saccharomyces genome database ID SGD:S000001121, a variant thereof or a fragment thereof), a IRE1 gene ortholog or a IRE1 gene paralog. In a specific embodiment, the second genetic modification can cause the modulation in the expression of a native KIN2 gene (and, in some embodiments bearing the Saccharomyces genome database SGD:S000004086, a variant thereof or a fragment thereof), a KIN2 gene ortholog or a KIN2 gene paralog and/or the expression of an heterologous polypeptide encoded by a KIN2 gene (and, in some embodiments bearing the Saccharomyces genome database ID SGD:S000004086, a variant thereof or a fragment thereof), a KIN2 gene ortholog or a KIN2 gene paralog.

The second genetic modification can be located in a gene encoding a protein exhibiting an oxidoreductase activity in the ER. The second genetic modification can cause the modulation in the expression (increasing or decreasing of the expression) of a native gene encoding a protein disulfide isomerase, a peptidyl-prolyl cis-trans isomerase or a thiol oxidase and/or can cause the expression of a heterologous gene encoding a protein disulfide isomerase, a peptidyl-prolyl cis-trans isomerase or a thiol oxidase. In some embodiments, the second genetic modification can cause the increase in the expression of a native gene encoding a protein disulfide isomerase, a peptidyl-prolyl cis-trans isomerase or a thiol oxidase and/or the increase in the expression of a heterologous gene encoding a protein disulfide isomerase, a peptidyl-prolyl cis-trans isomerase or a thiol oxidase. In a specific embodiment, the second genetic modification can cause the modulation in the expression of a native PDI1 gene (and, in some embodiments bearing the Saccharomyces genome database SGD:S000000548, a variant thereof or a fragment thereof), a PDI1 gene ortholog or a PDI1 gene paralog and/or the expression of an heterologous polypeptide encoded by a PDI1 gene (and, in some embodiments bearing the Saccharomyces genome database ID SGD:S000000548, a variant thereof or a fragment thereof), a PDI1 gene ortholog or a PDI1 gene paralog. In another specific embodiment, the second genetic modification can cause the overexpression of a native PDI1 gene (and, in some embodiments bearing the Saccharomyces genome database SGD:S000000548, a variant thereof or a fragment thereof), a PDI1 gene ortholog or a PDI1 gene paralog and/or the expression of an heterologous polypeptide encoded by a PDI1 gene (and, in some embodiments bearing the Saccharomyces genome database ID SGD:S000000548, a variant thereof or a fragment thereof), a PDI1 gene ortholog or a PDI1 gene paralog. In a specific embodiment, the second genetic modification can cause the modulation of the expression of a native CPR5 gene (and, in some embodiments bearing the Saccharomyces genome database SGD:S000002712, a variant thereof or a fragment thereof), a CPR5 gene ortholog or a CPR5 gene paralog and/or the expression of an heterologous polypeptide encoded by a CPR5 gene (and, in some embodiments bearing the Saccharomyces genome database ID SGD:S000002712, a variant thereof or a fragment thereof), a CPR5 gene ortholog or a CPR5 gene paralog. In a specific embodiment, the second genetic modification can cause the modulation in the expression of a native ERO1 gene (and, in some embodiments bearing the Saccharomyces genome database SGD:S000004599, a variant thereof or a fragment thereof), a ERO1 gene ortholog or a ERO1 gene paralog and/or the expression of an heterologous polypeptide encoded by a ERO1 gene (and, in some embodiments bearing the Saccharomyces genome database ID SGD:S000004599, a variant thereof or a fragment thereof), a ERO1 gene ortholog or a ERO1 gene paralog. In another specific embodiment, the second genetic modification can cause the overexpression of a native ERO1 gene (and, in some embodiments bearing the Saccharomyces genome database SGD:S000004599, a variant thereof or a fragment thereof), a ERO1 gene ortholog or a ERO1 gene paralog and/or the expression of an heterologous polypeptide encoded by a ERO1 gene (and, in some embodiments bearing the Saccharomyces genome database ID SGD:S000004599, a variant thereof or a fragment thereof), a ERO1 gene ortholog or a ERO1 gene paralog.

In some specific embodiments, the recombinant yeast host cell of the present disclosure comprises at least two second genetic modifications: one for the overexpression of a native PDI1 gene (and, in some embodiments bearing the Saccharomyces genome database SGD:S000000548, a variant thereof or a fragment thereof), a PDI1 gene ortholog or a PDI1 gene paralog and/or the expression of an heterologous polypeptide encoded by a PDI1 gene (and, in some embodiments bearing the Saccharomyces genome database ID SGD:S000000548, a variant thereof or a fragment thereof), a PDI1 gene ortholog or a PDI1 gene paralog and a further one for the overexpression of a native ERO1 gene (and, in some embodiments bearing the Saccharomyces genome database SGD:S000004599, a variant thereof or a fragment thereof), a ERO1 gene ortholog or a ERO1 gene paralog and/or the expression of an heterologous polypeptide encoded by a ERO1 gene (and, in some embodiments bearing the Saccharomyces genome database ID SGD:S000004599, a variant thereof or a fragment thereof), a ERO1 gene ortholog or a ERO1 gene paralog.

In some alternative embodiments, the recombinant yeast host cell of the present disclosure comprises at least three second genetic modifications: one for the overexpression of a native PDI1 gene (and, in some embodiments bearing the Saccharomyces genome database SGD:S000000548, a variant thereof or a fragment thereof), a PDI1 gene ortholog or a PDI1 gene paralog and/or the expression of an heterologous polypeptide encoded by a PDI1 gene (and, in some embodiments bearing the Saccharomyces genome database ID SGD:S000000548, a variant thereof or a fragment thereof), a PDI1 gene ortholog or a PDI1 gene paralog, a further one for the overexpression of a native ERO1 gene (and, in some embodiments bearing the Saccharomyces genome database SGD:S000004599, a variant thereof or a fragment thereof), a ERO1 gene ortholog or a ERO1 gene paralog and/or the expression of an heterologous polypeptide encoded by a ERO1 gene (and, in some embodiments bearing the Saccharomyces genome database ID SGD:S000004599, a variant thereof or a fragment thereof), a ERO1 gene ortholog or a ERO1 gene paralog and yet another one for the overexpression of a native KAR2 gene (and, in some embodiments bearing the Saccharomyces genome database SGD:S000003571, a variant thereof or a fragment thereof), a KAR2 gene ortholog or a KAR2 gene paralog and/or the expression of an heterologous polypeptide encoded by a KAR2 gene (and, in some embodiments bearing the Saccharomyces genome database ID SGD:S000003571, a variant thereof or a fragment thereof), a KAR2 gene ortholog or a KAR2 gene paralog.

The second genetic location can be located in a gene encoding a protein involved in the ER-associated degradation pathway (ERAD). In such embodiment, the second genetic modification can be the modulation in the expression (increasing or decreasing) of a gene encoding a protein involved in ERAD. In an embodiment, the second genetic modification can include the modulation in the expression of the native MNL1 gene (and, in some embodiments bearing the Saccharomyces genome database ID SGD:S000001247, a variant thereof or a fragment thereof), a MNL1 gene ortholog or a MNL1 gene paralog and/or the expression of an heterologous polypeptide encoded by a MNL1 gene (and, in some embodiments bearing the Saccharomyces genome database ID SGD:S000001247, a variant thereof or a fragment thereof), a MNL1 gene ortholog or a MNL1 gene paralog. In an embodiment, the second genetic modification can include the modulation in the expression of the native YOS9 gene (and, in some embodiments bearing the Saccharomyces genome database ID SGD:S000002464, a variant thereof or a fragment thereof), a YOS9 gene ortholog or a YOS9 gene paralog and/or the expression of an heterologous polypeptide encoded by a YOS9 gene (and, in some embodiments bearing the Saccharomyces genome database ID SGD:S000002464, a variant thereof or a fragment thereof), a YOS9 gene ortholog or a YOS9 gene paralog. In an embodiment, the second genetic modification can include the modulation in the expression of the native UBC7 gene (and, in some embodiments bearing the Saccharomyces genome database ID SGD:S000004624, a variant thereof or a fragment thereof), a UBC7 gene ortholog or a UBC7 gene paralog and/or the expression of an heterologous polypeptide encoded by a UBC7 gene (and, in some embodiments bearing the Saccharomyces genome database ID SGD:S000004624, a variant thereof or a fragment thereof), a UBC7 gene ortholog or a UBC7 gene paralog. In an embodiment, the second genetic modification can include the modulation in the expression of the native DER1 gene (and, in some embodiments bearing the Saccharomyces genome database ID SGD:S000000405, a variant thereof or a fragment thereof), a DER1 gene ortholog or a DER1 gene paralog and/or the expression of an heterologous polypeptide encoded by a DER1 gene (and, in some embodiments bearing the Saccharomyces genome database ID SGD:S000000405, a variant thereof or a fragment thereof), a DER1 gene ortholog or a DER1 gene paralog.

The second genetic modification can be located in a gene encoding a protein involved in the expansion of the ER. The second genetic modification can be the modulation in the expression of a gene encoding a protein involved in lipid biosynthesis (such as a transcription factor or a Mg2+-dependent phosphatidate (PA) phosphatase). In an embodiment, the second genetic modification can include the modulation in the expression of the native OPI1 gene (and, in some embodiments bearing the Saccharomyces genome database ID SGD:S000001012, a variant thereof or a fragment thereof), a OPI1 gene ortholog or a OPI1 gene paralog and/or the expression of an heterologous polypeptide encoded by a OPI1 gene (and, in some embodiments bearing the Saccharomyces genome database ID SGD:S000001012, a variant thereof or a fragment thereof), a OPI1 gene ortholog or a OPI1 gene paralog. In an embodiment, the second genetic modification can include the modulation in the expression of the native PAH1 gene (and, in some embodiments bearing the Saccharomyces genome database ID SGD:S000004775, a variant thereof or a fragment thereof), a PAH1 gene ortholog or a PAH1 gene paralog and/or the expression of an heterologous polypeptide encoded by a PAH1 gene (and, in some embodiments bearing the Saccharomyces genome database ID SGD:S000004775, a variant thereof or a fragment thereof), a PAH1 gene ortholog or a PAH1 gene paralog.

The second genetic modification can be located in a gene encoding a protein involved in protein trafficking. The second genetic modification can be located in a gene encoding a protein involved in the formation, maintenance, trafficking and/or fusion of COPII vesicles. The second genetic modification can cause the modulation in the expression (increasing or decreasing the expression) of a native gene encoding a protein involved in the formation, maintenance, trafficking and/or fusion of COPII vesicles and/or can cause the expression of a heterologous gene encoding a protein in the formation, maintenance and trafficking of COPII vesicles. In a specific embodiment, the second genetic modification can cause the modulation in the expression of a native SARI gene (and, in some embodiments bearing the Saccharomyces genome database SGD:S000006139, a variant thereof or a fragment thereof), a SARI gene ortholog or a SARI gene paralog and/or the expression of an heterologous polypeptide encoded by a SARI gene (and, in some embodiments bearing the Saccharomyces genome database ID SGD:S000006139, a variant thereof or a fragment thereof), a SARI gene ortholog or a SARI gene paralog. In a specific embodiment, the second genetic modification can cause the modulation in the expression of a native SEC16 gene (and, in some embodiments bearing the Saccharomyces genome database SGD:S000006006, a variant thereof or a fragment thereof), a SEC16 gene ortholog or a SEC16 gene paralog and/or the expression of an heterologous polypeptide encoded by a SEC16 gene (and, in some embodiments bearing the Saccharomyces genome database ID SGD:S000006006, a variant thereof or a fragment thereof), a SEC16 gene ortholog or a SEC16 gene paralog. In a specific embodiment, the second genetic modification can cause the modulation in the expression of a native SBH1 gene (and, in some embodiments bearing the Saccharomyces genome database SGD:S000002128, a variant thereof or a fragment thereof), a SBH1 gene ortholog or a SBH1 gene paralog and/or the expression of an heterologous polypeptide encoded by a SBH1 gene (and, in some embodiments bearing the Saccharomyces genome database ID SGD:S000002128, a variant thereof or a fragment thereof), a SBH1 gene ortholog or a SBH1 gene paralog. In a specific embodiment, the second genetic modification can cause the modulation in the expression of a native SEC12 gene (and, in some embodiments bearing the Saccharomyces genome database SGD:S000005309, a variant thereof or a fragment thereof), a SEC12 gene ortholog or a SEC12 gene paralog and/or the expression of an heterologous polypeptide encoded by a SEC12 gene (and, in some embodiments bearing the Saccharomyces genome database ID SGD:S000005309, a variant thereof or a fragment thereof), a SEC12 gene ortholog or a SEC12 gene paralog. In a specific embodiment, the second genetic modification can cause the modulation in the expression of a native SEC23 gene (and, in some embodiments bearing the Saccharomyces genome database SGD:S000006385, a variant thereof or a fragment thereof), a SEC23 gene ortholog or a SEC23 gene paralog and/or the expression of an heterologous polypeptide encoded by a SEC23 gene (and, in some embodiments bearing the Saccharomyces genome database ID SGD:S000006385, a variant thereof or a fragment thereof), a SEC23 gene ortholog or a SEC23 gene paralog. In a specific embodiment, the second genetic modification can cause the modulation in the expression of a native SEC24 gene (and, in some embodiments bearing the Saccharomyces genome database SGD:S000001371, a variant thereof or a fragment thereof), a SEC24 gene ortholog or a SEC24 gene paralog and/or the expression of an heterologous polypeptide encoded by a SEC24 gene (and, in some embodiments bearing the Saccharomyces genome database ID SGD:S000001371, a variant thereof or a fragment thereof), a SEC24 gene ortholog or a SEC24 gene paralog. In a specific embodiment, the second genetic modification can cause the modulation in the expression of a native SEC13 gene (and, in some embodiments bearing the Saccharomyces genome database SGD:S000004198, a variant thereof or a fragment thereof), a SEC13 gene ortholog or a SEC13 gene paralog and/or the expression of an heterologous polypeptide encoded by a SEC13 gene (and, in some embodiments bearing the Saccharomyces genome database ID SGD:S000004198, a variant thereof or a fragment thereof), a SEC13 gene ortholog or a SEC13 gene paralog. In a specific embodiment, the second genetic modification can cause the modulation in the expression of a native SEC31 gene (and, in some embodiments bearing the Saccharomyces genome database SGD:S000002354, a variant thereof or a fragment thereof), a SEC31 gene ortholog or a SEC31 gene paralog and/or the expression of an heterologous polypeptide encoded by a SEC31 gene (and, in some embodiments bearing the Saccharomyces genome database ID SGD:S000002354, a variant thereof or a fragment thereof), a SEC31 gene ortholog or a SEC31 gene paralog. In a specific embodiment, the second genetic modification can cause the modulation in the expression of a native COG5 gene (and, in some embodiments bearing the Saccharomyces genome database SGD:S000004996, a variant thereof or a fragment thereof), a COG5 gene ortholog or a COG5 gene paralog and/or the expression of an heterologous polypeptide encoded by a COG5 gene (and, in some embodiments bearing the Saccharomyces genome database ID SGD:S000004996, a variant thereof or a fragment thereof), a COG5 gene ortholog or a COG5 gene paralog. In a specific embodiment, the second genetic modification can cause the modulation in the expression of a native BOS1 gene (and, in some embodiments bearing the Saccharomyces genome database SGD:S000004068, a variant thereof or a fragment thereof), a BOS1 gene ortholog or a BOS1 gene paralog and/or the expression of an heterologous polypeptide encoded by a BOS1 gene (and, in some embodiments bearing the Saccharomyces genome database ID SGD:S000004068, a variant thereof or a fragment thereof), a BOS1 gene ortholog or a BOS1 gene paralog. In a specific embodiment, the second genetic modification can cause the modulation in the expression of a native COG6 gene (and, in some embodiments bearing the Saccharomyces genome database SGD:S000004986, a variant thereof or a fragment thereof), a COG6 gene ortholog or a COG6 gene paralog and/or the expression of an heterologous polypeptide encoded by a COG6 gene (and, in some embodiments bearing the Saccharomyces genome database ID SGD:S000004986, a variant thereof or a fragment thereof), a COG6 gene ortholog or a COG6 gene paralog. In a specific embodiment, the second genetic modification can cause the modulation in the expression of a native COY1 gene (and, in some embodiments bearing the Saccharomyces genome database SGD:S000001662, a variant thereof or a fragment thereof), a COY1 gene ortholog or a COY1 gene paralog and/or the expression of an heterologous polypeptide encoded by a COY1 gene (and, in some embodiments bearing the Saccharomyces genome database ID SGD:S000001662, a variant thereof or a fragment thereof), a COY1 gene ortholog or a COY1 gene paralog. In a specific embodiment, the second genetic modification can cause the modulation in the expression of a native SLY1 gene (and, in some embodiments bearing the Saccharomyces genome database SGD:S000002597, a variant thereof or a fragment thereof), a SLY1 gene ortholog or a SLY1 gene paralog and/or the expression of an heterologous polypeptide encoded by a SLY1 gene (and, in some embodiments bearing the Saccharomyces genome database ID SGD:S000002597, a variant thereof or a fragment thereof), a SLY1 gene ortholog or a SLY1 gene paralog. In a specific embodiment, the second genetic modification can cause the modulation in the expression of a native ERV25 gene (and, in some embodiments bearing the Saccharomyces genome database SGD:S000004473, a variant thereof or a fragment thereof), a ERV25 gene ortholog or a ERV25 gene paralog and/or the expression of an heterologous polypeptide encoded by a ERV25 gene (and, in some embodiments bearing the Saccharomyces genome database ID SGD:S000004473, a variant thereof or a fragment thereof), a ERV25 gene ortholog or a ERV25 gene paralog. In a specific embodiment, the second genetic modification can cause the modulation in the expression of a native ERV29 gene (and, in some embodiments bearing the Saccharomyces genome database SGD:S000003516, a variant thereof or a fragment thereof), a ERV29 gene ortholog or a ERV29 gene paralog and/or the expression of an heterologous polypeptide encoded by a ERV29 gene (and, in some embodiments bearing the Saccharomyces genome database ID SGD:S000003516, a variant thereof or a fragment thereof), a ERV29 gene ortholog or a ERV29 gene paralog.

The second genetic modification can be located in a gene encoding a protein involved in the formation, maintenance and trafficking of COPI vesicles. The second genetic modification can cause the modulation in the expression (increasing or decreasing the expression) of a native gene encoding a protein involved in the formation, maintenance and trafficking of COPI vesicles. In an embodiment, the second genetic modification can include the modulation in the expression of the native GOS1 gene (and, in some embodiments bearing the Saccharomyces genome database ID SGD:S000001023, a variant thereof or a fragment thereof), a GOS1 gene ortholog or a GOS1 gene paralog and/or the expression of an heterologous polypeptide encoded by a GOS1 gene (and, in some embodiments bearing the Saccharomyces genome database ID SGD:S000001023, a variant thereof or a fragment thereof), a GOS1 gene ortholog or a GOS1 gene paralog.

The second genetic modification can be located in a gene encoding a protein involved in the formation, maintenance, trafficking and/or fusion of retromer complexes. The second genetic modification can cause the modulation in the expression (increasing or decreasing the expression) of a native gene encoding a protein involved in the formation, maintenance, trafficking and/or fusion of retromer complexes. In an embodiment, the second genetic modification can include the modulation in the expression of the native LAM1 gene (and, in some embodiments bearing the Saccharomyces genome database ID SGD:S000001198, a variant thereof or a fragment thereof), a LAM1 gene ortholog or a LAM1 gene paralog and/or the expression of an heterologous polypeptide encoded by a LAM1 gene (and, in some embodiments bearing the Saccharomyces genome database ID SGD:S000001198, a variant thereof or a fragment thereof), a LAM1 gene ortholog or a LAM1 gene paralog. In an embodiment, the second genetic modification can include the modulation in the expression of the native VPS5 gene (and, in some embodiments bearing the Saccharomyces genome database ID SGD:S000005595, a variant thereof or a fragment thereof), a VPS5 gene ortholog or a VPS5 gene paralog and/or the expression of an heterologous polypeptide encoded by a VPS5 gene (and, in some embodiments bearing the Saccharomyces genome database ID SGD:S000005595, a variant thereof or a fragment thereof), a VPS5 gene ortholog or a VPS5 gene paralog. In another embodiment, the second genetic modification can include the inactivation of the native VPS5 gene (and, in some embodiments bearing the Saccharomyces genome database ID SGD:S000005595, a variant thereof or a fragment thereof), a VPS5 gene ortholog or a VPS5 gene paralog. In an embodiment, the second genetic modification can include the modulation in the expression of the native VPS17 gene (and, in some embodiments bearing the Saccharomyces genome database ID SGD:S000005658, a variant thereof or a fragment thereof), a VPS17 gene ortholog or a VPS17 gene paralog and/or the expression of an heterologous polypeptide encoded by a VPS17 gene (and, in some embodiments bearing the Saccharomyces genome database ID SGD:S000005658, a variant thereof or a fragment thereof), a VPS17 gene ortholog or a VPS17 gene paralog. In another embodiment, the second genetic modification can include the inactivation of the native VPS17 gene (and, in some embodiments bearing the Saccharomyces genome database ID SGD:S000005658, a variant thereof or a fragment thereof), a VPS17 gene ortholog or a VPS17 gene paralog. In an embodiment, the second genetic modification can include the modulation in the expression of the native VPS26 gene (and, in some embodiments bearing the Saccharomyces genome database ID SGD:S000003589, a variant thereof or a fragment thereof), a VPS26 gene ortholog or a VPS26 gene paralog and/or the expression of an heterologous polypeptide encoded by a VPS26 gene (and, in some embodiments bearing the Saccharomyces genome database ID SGD:S000003589, a variant thereof or a fragment thereof), a VPS26 gene ortholog or a VPS26 gene paralog. In an embodiment, the second genetic modification can include the modulation in the expression of the native VPS29 gene (and, in some embodiments bearing the Saccharomyces genome database SGD:S000001054, a variant thereof or a fragment thereof), a VPS29 gene ortholog or a VPS29 gene paralog and/or the expression of an heterologous polypeptide encoded by a VPS29 gene (and, in some embodiments bearing the Saccharomyces genome database ID SGD:S000001054, a variant thereof or a fragment thereof), a VPS29 gene ortholog or a VPS29 gene paralog. In an embodiment, the second genetic modification can include the modulation in the expression of the native VPS35 gene (and, in some embodiments bearing the Saccharomyces genome database ID SGD:S000003690, a variant thereof or a fragment thereof), a VPS35 gene ortholog or a VPS35 gene paralog and/or the expression of an heterologous polypeptide encoded by a VPS35 gene (and, in some embodiments bearing the Saccharomyces genome database ID SGD:S000003690, a variant thereof or a fragment thereof), a VPS35 gene ortholog or a VPS35 gene paralog.

In some embodiments, the recombinant yeast host cell comprises at least two second genetic modifications: a first one causing the inactivation of the native VPS5 gene (and, in some embodiments bearing the Saccharomyces genome database ID SGD:S000005595, a variant thereof or a fragment thereof), a VPS5 gene ortholog or a VPS5 gene paralog and a further one causing the inactivation of the native VPS17 gene (and, in some embodiments bearing the Saccharomyces genome database ID SGD:S000005658, a variant thereof or a fragment thereof), a VPS17 gene ortholog or a VPS17 gene paralog. In some further embodiments, the recombinant yeast host cell comprises at least three second genetic modifications: a first one causing the inactivation of the native VPS5 gene (and, in some embodiments bearing the Saccharomyces genome database ID SGD:S000005595, a variant thereof or a fragment thereof), a VPS5 gene ortholog or a VPS5 gene paralog, a further one for the overexpression of a native PDI1 gene (and, in some embodiments bearing the Saccharomyces genome database SGD:S000000548, a variant thereof or a fragment thereof), a PDI1 gene ortholog or a PDI1 gene paralog and/or the expression of an heterologous polypeptide encoded by a PDI1 gene (and, in some embodiments bearing the Saccharomyces genome database ID SGD:S000000548, a variant thereof or a fragment thereof), a PDI1 gene ortholog or a PDI1 gene paralog and a yet further one for the overexpression of a native ERO1 gene (and, in some embodiments bearing the Saccharomyces genome database SGD:S000004599, a variant thereof or a fragment thereof), a ERO1 gene ortholog or a ERO1 gene paralog and/or the expression of an heterologous polypeptide encoded by a ERO1 gene (and, in some embodiments bearing the Saccharomyces genome database ID SGD:S000004599, a variant thereof or a fragment thereof), a ERO1 gene ortholog or a ERO1 gene paralog. In some further embodiments, the recombinant yeast host cell comprises at least three second genetic modifications: a first one causing the inactivation of the native VPS17 gene (and, in some embodiments bearing the Saccharomyces genome database ID SGD:S000005658, a variant thereof or a fragment thereof), a VPS17 gene ortholog or a VPS17 gene paralog, a further one for the overexpression of a native PDI1 gene (and, in some embodiments bearing the Saccharomyces genome database SGD:S000000548, a variant thereof or a fragment thereof), a PDI1 gene ortholog or a PDI1 gene paralog and/or the expression of an heterologous polypeptide encoded by a PDI1 gene (and, in some embodiments bearing the Saccharomyces genome database ID SGD:S000000548, a variant thereof or a fragment thereof), a PDI1 gene ortholog or a PDI1 gene paralog and a yet further one for the overexpression of a native ERO1 gene (and, in some embodiments bearing the Saccharomyces genome database SGD:S000004599, a variant thereof or a fragment thereof), a ERO1 gene ortholog or a ERO1 gene paralog and/or the expression of an heterologous polypeptide encoded by a ERO1 gene (and, in some embodiments bearing the Saccharomyces genome database ID SGD:S000004599, a variant thereof or a fragment thereof), a ERO1 gene ortholog or a ERO1 gene paralog.

The second genetic modification can be located in a gene encoding a protein involved in the formation, maintenance, trafficking and/or fusion of secretory vesicles (trafficking between the Golgi system and the membrane). The second genetic modification can cause the modulation in the expressing (increasing or decreasing the expression) of a native gene encoding a protein involved in the formation, maintenance and trafficking of secretory vesicles and/or can cause the expression of a heterologous gene encoding a protein in the formation, maintenance, trafficking and/or fusion of secretory vesicles. In a specific embodiment, the second genetic modification can cause the modulation in the expression of a native SSO1 gene (and, in some embodiments bearing the Saccharomyces genome database SGD:S000006153, a variant thereof or a fragment thereof), a SSO1 gene ortholog or a SSO1 gene paralog and/or the expression of an heterologous polypeptide encoded by a SSO1 gene (and, in some embodiments bearing the Saccharomyces genome database ID SGD:S000006153, a variant thereof or a fragment thereof), a SSO1 gene ortholog or a SSO1 gene paralog. In a specific embodiment, the second genetic modification can cause the modulation in the expression of a native SSO2 gene (and, in some embodiments bearing the Saccharomyces genome database SGD:S000004795, a variant thereof or a fragment thereof), a SSO2 gene ortholog or a SSO2 gene paralog and/or the expression of an heterologous polypeptide encoded by a SSO2 gene (and, in some embodiments bearing the Saccharomyces genome database ID SGD:S000004795, a variant thereof or a fragment thereof), a SSO2 gene ortholog or a SSO2 gene paralog. In a specific embodiment, the second genetic modification can cause the modulation in the expression of a native SNC2 gene (and, in some embodiments bearing the Saccharomyces genome database SGD:S000005854, a variant thereof or a fragment thereof), a SNC2 gene ortholog or a SNC2 gene paralog and/or the expression of an heterologous polypeptide encoded by a SNC2 gene (and, in some embodiments bearing the Saccharomyces genome database ID SGD:S000005854, a variant thereof or a fragment thereof), a SNC2 gene ortholog or a SNC2 gene paralog. In a specific embodiment, the second genetic modification can cause the modulation in the expression of a native SEC1 gene (and, in some embodiments bearing the Saccharomyces genome database SGD:S000002571, a variant thereof or a fragment thereof), a SEC1 gene ortholog or a SEC1 gene paralog and/or the expression of an heterologous polypeptide encoded by a SEC1 gene (and, in some embodiments bearing the Saccharomyces genome database ID SGD:S000002571, a variant thereof or a fragment thereof), a SEC1 gene ortholog or a SEC1 gene paralog. In a specific embodiment, the second genetic modification can cause the modulation in the expression of a native EX070 gene (and, in some embodiments bearing the Saccharomyces genome database SGD:S000003621, a variant thereof or a fragment thereof), a EX070 gene ortholog or a EX070 gene paralog and/or the expression of an heterologous polypeptide encoded by a EX070 gene (and, in some embodiments bearing the Saccharomyces genome database ID SGD:S000003621, a variant thereof or a fragment thereof), a EX070 gene ortholog or a EX070 gene paralog. In a specific embodiment, the second genetic modification can cause the modulation in the expression of a native YPT32 gene (and, in some embodiments bearing the Saccharomyces genome database SGD:S000003178, a variant thereof or a fragment thereof), a YPT32 gene ortholog or a YPT32 gene paralog and/or the expression of an heterologous polypeptide encoded by a YPT32 gene (and, in some embodiments bearing the Saccharomyces genome database ID SGD:S000003178, a variant thereof or a fragment thereof), a YPT32 gene ortholog or a YPT32 gene paralog. In a specific embodiment, the second genetic modification can cause the modulation in the expression of a native SEC4 gene (and, in some embodiments bearing the Saccharomyces genome database SGD:S000001889, a variant thereof or a fragment thereof), a SEC4 gene ortholog or a SEC4 gene paralog and/or the expression of an heterologous polypeptide encoded by a SEC4 gene (and, in some embodiments bearing the Saccharomyces genome database ID SGD:S000001889, a variant thereof or a fragment thereof), a SEC4 gene ortholog or a SEC4 gene paralog.

The second genetic location can be located in a gene encoding a protein involved in the vacuolar pathway. In such embodiment, the second genetic modification can be the modulation in the expression (increasing or decreasing the expression) of a gene encoding a protein involved in the vacuolar pathway (which can be, for example, a vacuolar protease, a vacuolar protease activator, a component involved in trafficking towards vacuoles and/or in autophagy). In an embodiment, the second genetic modification can include the modulation in the expression of the native PRB1 gene (and, in some embodiments bearing the Saccharomyces genome database ID SGD:S000000786, a variant thereof or a fragment thereof), a PRB1 gene ortholog or a PRB1 gene paralog and/or the expression of an heterologous polypeptide encoded by a PRB1 gene (and, in some embodiments bearing the Saccharomyces genome database ID SGD:S000000786, a variant thereof or a fragment thereof), a PRB1 gene ortholog or a PRB1 gene paralog. In another embodiment, the second genetic modification can include the inactivation the native PRB1 gene (and, in some embodiments bearing the Saccharomyces genome database ID SGD:S000000786, a variant thereof or a fragment thereof), a PRB1 gene ortholog or a PRB1 gene paralog. In an embodiment, the second genetic modification can include the modulation in the expression of the native VMA3 gene (and, in some embodiments bearing the Saccharomyces genome database ID SGD:S000000753, a variant thereof or a fragment thereof), a VMA3 gene ortholog or a VMA3 gene paralog and/or the expression of an heterologous polypeptide encoded by a VMA3 gene (and, in some embodiments bearing the Saccharomyces genome database ID SGD:S000000753, a variant thereof or a fragment thereof), a VMA3 gene ortholog or a VMA3 gene paralog. In an embodiment, the second genetic modification can include the modulation of the expression of the native VPS8 gene (and, in some embodiments bearing the Saccharomyces genome database ID SGD:S000000002, a variant thereof or a fragment thereof), a VPS8 gene ortholog or a VPS8 gene paralog and/or the expression of an heterologous polypeptide encoded by a VPS8 gene (and, in some embodiments bearing the Saccharomyces genome database ID SGD:S000000002, a variant thereof or a fragment thereof), a VPS8 gene ortholog or a VPS8 gene paralog. In an embodiment, the second genetic modification can include the modulation in the expression of the native VPS21 gene (and, in some embodiments bearing the Saccharomyces genome database ID SGD:S000005615, a variant thereof or a fragment thereof), a VPS21 gene ortholog or a VPS21 gene paralog and/or the expression of an heterologous polypeptide encoded by a VPS21 gene (and, in some embodiments bearing the Saccharomyces genome database ID SGD:S000005615, a variant thereof or a fragment thereof), a VPS21 gene ortholog or a VPS21 gene paralog. In an embodiment, the second genetic modification can include the modulation in the expression of the native MTC6 gene (and, in some embodiments bearing the Saccharomyces genome database ID SGD:S000001194, a variant thereof or a fragment thereof), a MTC6 gene ortholog or a MTC6 gene paralog and/or the expression of an heterologous polypeptide encoded by a MTC6 gene (and, in some embodiments bearing the Saccharomyces genome database ID SGD:S000001194, a variant thereof or a fragment thereof), a MTC6 gene ortholog or a MTC6 gene paralog. In a specific embodiment, the second genetic modification can cause the modulation in the expression of a native SEC4 gene (and, in some embodiments bearing the Saccharomyces genome database SGD:S000001889, a variant thereof or a fragment thereof), a SEC4 gene ortholog or a SEC4 gene paralog and/or the expression of an heterologous polypeptide encoded by a SEC4 gene (and, in some embodiments bearing the Saccharomyces genome database ID SGD:S000001889, a variant thereof or a fragment thereof), a SEC4 gene ortholog or a SEC4 gene paralog.

In some embodiments, the recombinant yeast host cell comprises at least two second genetic modifications: a first one causing the inactivation the native PRB1 gene (and, in some embodiments bearing the Saccharomyces genome database ID SGD:S000000786, a variant thereof or a fragment thereof), a PRB1 gene ortholog or a PRB1 gene paralog, and a further one causing the inactivation of the native VPS5 gene (and, in some embodiments bearing the Saccharomyces genome database ID SGD:S000005595, a variant thereof or a fragment thereof), a VPS5 gene ortholog or a VPS5 gene paralog. In some embodiments, the recombinant yeast host cell comprises at least three second genetic modifications: a first one causing the inactivation the native PRB1 gene (and, in some embodiments bearing the Saccharomyces genome database ID SGD:S000000786, a variant thereof or a fragment thereof), a PRB1 gene ortholog or a PRB1 gene paralog, a further one for the overexpression of a native PDI1 gene (and, in some embodiments bearing the Saccharomyces genome database SGD:S000000548, a variant thereof or a fragment thereof), a PDI1 gene ortholog or a PDI1 gene paralog and/or the expression of an heterologous polypeptide encoded by a PDI1 gene (and, in some embodiments bearing the Saccharomyces genome database ID SGD:S000000548, a variant thereof or a fragment thereof), a PDI1 gene ortholog or a PDI1 gene paralog and yet a further one for the overexpression of a native ERO1 gene (and, in some embodiments bearing the Saccharomyces genome database SGD:S000004599, a variant thereof or a fragment thereof), a ERO1 gene ortholog or a ERO1 gene paralog and/or the expression of an heterologous polypeptide encoded by a ERO1 gene (and, in some embodiments bearing the Saccharomyces genome database ID SGD:S000004599, a variant thereof or a fragment thereof), a ERO1 gene ortholog or a ERO1 gene paralog.

The second genetic modification can be located in a gene encoding a protein involved in the cell wall stability pathway. The second genetic modification can be the modulation in the expression (increasing or decreasing) of a gene encoding a protein involved in providing cell wall stability. In an embodiment, the second genetic modification can include the modulation in the expression of the native CCW12 gene (and, in some embodiments bearing the Saccharomyces genome database ID SGD:S000004100, a variant thereof or a fragment thereof), a CCW12 gene ortholog or a CCW12 gene paralog and/or the expression of an heterologous polypeptide encoded by a CCW12 gene (and, in some embodiments bearing the Saccharomyces genome database ID SGD:S000004100, a variant thereof or a fragment thereof), a CCW12 gene ortholog or a CCW12 gene paralog. In an embodiment, the second genetic modification can include the modulation in the expression of the native CWP2 gene (and, in some embodiments bearing the Saccharomyces genome database ID SGD:S000001956, a variant thereof or a fragment thereof), a CWP2 gene ortholog or a CWP2 gene paralog and/or the expression of an heterologous polypeptide encoded by a CWP2 gene (and, in some embodiments bearing the Saccharomyces genome database ID SGD:S000001956, a variant thereof or a fragment thereof), a CWP2 gene ortholog or a CWP2 gene paralog. In an embodiment, the second genetic modification can include the modulation in the expression of the native SED1 gene (and, in some embodiments bearing the Saccharomyces genome database ID SGD:S000002484, a variant thereof or a fragment thereof), a SED1 gene ortholog or a SED1 gene paralog and/or the expression of an heterologous polypeptide encoded by a SED1 gene (and, in some embodiments bearing the Saccharomyces genome database ID SGD:S000002484, a variant thereof or a fragment thereof), a SED1 gene ortholog or a SED1 gene paralog.

In an embodiment, the second genetic modification can include the modulation in the expression of the native BMH2 gene (and, in some embodiments bearing the Saccharomyces genome database ID SGD:S000002506, a variant thereof or a fragment thereof), a BMH2 gene ortholog or a BMH2 gene paralog and/or the expression of an heterologous polypeptide encoded by a BMH2 gene (and, in some embodiments bearing the Saccharomyces genome database ID SGD:S000002506, a variant thereof or a fragment thereof), a BMH2 gene ortholog or a BMH2 gene paralog.

In an embodiment, the second genetic modification can include the modulation in the expression of the native KEX2 gene (and, in some embodiments bearing the Saccharomyces genome database ID SGD:S000005182, a variant thereof or a fragment thereof), a BMH2 gene ortholog or a KEX2 gene paralog and/or the expression of an heterologous polypeptide encoded by a KEX2 gene (and, in some embodiments bearing the Saccharomyces genome database ID SGD:S000005182, a variant thereof or a fragment thereof), a KEX2 gene ortholog or a KEX2 gene paralog.

### Recombinant Yeast Host Cell

The present disclosure concerns recombinant yeast host cells that have been genetically engineered to express a heterologous polypeptide or to over-express a native polypeptide. The recombinant yeast host cells of the present disclosure are intended for use in the production of the heterologous polypeptide or the native polypeptide. In one embodiment, the recombinant yeast host cell is intended for use in the production of polypeptides intended to be secreted or exported outside the recombinant yeast host cell.

In the context of the present disclosure, when recombinant yeast cell is qualified as being “genetically engineered”, it is understood to mean that it has been manipulated to add and/or remove at least one or more heterologous or exogenous nucleic acid residue. In some embodiments, the one or more nucleic acid residues that are added can be derived from an heterologous cell or the recombinant host cell itself. In the latter scenario, the nucleic acid residue(s) is (are) added at one or more genomic location which is different than the native genomic location. The genetic manipulations did not occur in nature and are the results of in vitro manipulations of the yeast.

The present disclosure concerns recombinant yeast host cells that have been genetically engineered and include a first and a second genetic modification. The genetic modification(s) that is(are) aimed at increasing the expression of a specific targeted gene (which is considered heterologous or native to the yeast host cell) and can be made in one or multiple (e.g., 1, 2, 3, 4, 5, 6, 7, 8 or more) genetic locations. In some embodiments, the one or more nucleic acid residues that are added can be derived from an heterologous cell or the recombinant yeast host cell itself. In the latter scenario, the nucleic acid residue(s) is (are) added at one or more genomic location which is different than the native genomic location. The genetic manipulations did not occur in nature and are the results of in vitro manipulations of the yeast. In some embodiments, the genetic modification(s) in the recombinant yeast host cell consist essentially of or consist of a genetic modification allowing the expression of an heterologous nucleic acid molecule encoding an heterologous polypeptide. The genetic modification(s) that is (are) aimed at reducing the expression of a specific targeted gene (which is considered native to the yeast host cell) can be made in one or more copies of the native gene.

In the context of the present disclosure, the recombinant host cell is a yeast. Suitable recombinant yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces, Torula, Hanseniaspora, Lachancea, Wickerhamomyces or Yarrowia. Suitable yeast species can include, for example, S. cerevisiae, S. bulderi, S. barnetti, S. exiguus, S. uvarum, S. diastaticus, C. utilis, K. lactis, K. marxianus K. fragilis, Hanseniaspora vineae, Lachancea fermentati, Lachancea thermotolerans, Schizosaccharomyces japonicus and/or Wickerhamomyces anomalus. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiment, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiment, the host cell can be an oleaginous microalgae host cell (e.g., for example, from the genus Thraustochytrium or Schizochytrium). In an embodiment, the recombinant yeast host cell is from the genus Saccharomyces and, in some embodiments, from the species Saccharomyces cerevisiae. In an embodiment, the recombinant yeast host cell is from the genus Hansenula sp. and, in some embodiments from the species Hansenula polymorpha. In an embodiment, the recombinant yeast host cell is from the genus Pichia sp. and, in some embodiments from the species Pichia pastoris. In an embodiment, the recombinant yeast host cell is from the genus Yarrowia sp. and, in some embodiments from the species Yarrowia lipolytica. In an embodiment, the recombinant yeast host cell is from the genus Kluyveromyces sp. and, in some embodiments from the species Kluyveromyces lactis. In an embodiment, the recombinant yeast host cell is from the genus Pichia sp. and, in some embodiments from the species Pichia pastoris.

### Propagating Recombinant Yeast Host Dell and Making Yeast Products

The present disclosure allows for the propagation of recombinant yeast host cell of the present disclosure and ultimately the secretion of the overexpressed native polypeptide or the heterologous polypeptide (associated with the first genetic modification). In the propagation process, the recombinant yeast host cell is placed in a culture medium under suitable condition for growth. Since the second genetic modification is intended to restore, at least in part, the growth rate of the recombinant yeast host cell when compared to the parental yeast host cell and/or to increase the growth rate of the recombinant yeast host cell when compared to the control yeast host cell, the propagating process can be conducted so as to restore and/or increase the growth rate of the recombinant yeast host cell. In the propagating process of the present disclosure, the recombinant yeast host cell can an increase in growth rate of at least 100, 105, 110, 115, 120, 125, 130, 135, 140, 145, 150, 155, 160, 165, 170, 175, 180, 185, 190, 195, 200, 205, 210, 215, 200% or more when compared to the growth rate of the control yeast host cell (determined under similar conditions). This increase in growth rate is observed when one compares the growth rate of the control yeast host cell which expresses the heterologous polypeptide or the native polypeptide (in the presence or in the absence of the signal sequence) but lacks the second genetic modification. In one embodiment, this increase in growth rate is observed when one compares the growth rate of a control yeast host cell which expresses the first genetic modification together with a signal sequence (e.g., the first genetic modification).

The culture medium using during the propagation can comprise a carbon source (such as, for example, molasses, sucrose, glucose, dextrose syrup, ethanol, corn, glycerol, corn steep liquor and/or a lignocellulosic biomass), a nitrogen source (such as, for example, ammonia or another inorganic source of nitrogen) and a phosphorous source (such as, for example, phosphoric acid or another inorganic source of phosphorous). The culture medium can further comprises additional micronutrients such as vitamins and/or minerals to support the propagation of the recombinant yeast host cell.

The propagation process can be conducted at a specific pH and/or a specific temperature which is optimal for the expression of the heterologous polypeptide or for the over-expression of the native polypeptide. As such, in embodiments in which the yeast is from the genus Saccharomyces, the process can comprise controlling the pH of the culture medium to between about 3.0 to about 6.0, about 3.5 to about 5.5 or about 4.0 to about 5.5. In a specific embodiment, the pH is controlled at about 4.5. In another example, in embodiments in which the yeast is from the genus Saccharomyces, the process can comprise controlling the temperature of the culture medium between about 20° C. to about 40° C., about 25° C. to about 30° C. or about 30° C. to about 35° C. In a specific embodiment, the temperature is controlled at between about 30° C. to about 35° C. (32° C. for example).

In some embodiments, the propagation process is conducted under aerobic conditions.

In some embodiments, the heterologous polypeptide or the native polypeptide is expressed and secreted to the extracellular space in a free form (e.g., not physically associated with the recombinant yeast host cell). In such embodiment, following propagation of the recombinant yeast host cells, the heterologous or native polypeptide are produced and secreted in the culture medium. The process can also comprise substantially isolating and optionally purifying the heterologous or native polypeptide from the culture medium. As used in the context of the present disclosure, the expression “substantially isolating and optionally purifying the heterologous or native polypeptide from the culture medium” refers to the removal of the majority of the components (such as the propagated recombinant yeast host cell) of culture medium from the heterologous or native polypeptide and providing same in an isolated/purified form. The heterologous or native polypeptide can be provided in a liquid form or in a solid (dried) form. As such, the present disclosure provides an isolated heterologous or native polypeptide obtainable or obtained by the process described herein.

In some embodiments, the heterologous polypeptide or the native polypeptide is expressed and secreted in a cell-associated form and is associated to the cell wall or cell membrane of the recombinant yeast host cell. In such embodiment, the heterologous or native polypeptide accumulates on the surface of the propagated recombinant yeast host cells. The process can include substantially purifying the propagated recombinant yeast host cells from the culture medium. The process can also include a step of subsequent step lysing and/or drying the propagated yeast host cells. The process can also comprise separating, at least in part, the heterologous or native polypeptide from the propagated recombinant yeast host cell. The process can also include a step of subsequent step drying the separated heterologous or native polypeptide.

For example, in the process of the present disclosure, the recombinant yeast host cells can be lysed using autolysis (which can be optionally be performed in the presence of additional exogenous enzymes) or homogenized (for example using a bead-milling technique). In an embodiment, the propagated recombinant yeast host cells can be lysed using autolysis. In some embodiments, the propagated recombinant cells can be submitted to a combined heat and pH treatment for a specific amount of time (e.g., 24 h) in order to cause the autolysis of the propagated recombinant yeast host cells to provide the lysed recombinant yeast host cells. For example, the propagated recombinant cells can be submitted to a temperature of between about 40° C. to about 70 ° C. or between about 50° C. to about 60° C. The propagated recombinant cells can be submitted to a temperature of at least about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C. or 70° C. Alternatively or in combination the propagated recombinant cells can be submitted to a temperature of no more than about 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., 47° C., 46° C., 45° C., 44° C., 43° C., 42° C., 41° C. or 40° C. In another example, the propagated recombinant cells can be submitted to a pH between about 4.0 and 8.5 or between about 5.0 and 7.5. The propagated recombinant cells can be submitted to a pH of at least about, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5. Alternatively or in combination, the propagated recombinant cells can be submitted to a pH of no more than 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3., 5.2, 5.1, 5.0, 4.9, 4.8, 4.7, 4.6 or 4.5.

The process of the present disclosure can include, in some embodiments, a step of partially inactivating the propagated yeast host cells. As used in the context of the present disclosure, the expression “partial inactivation” refers to the fact the viability of a proportion of the propagated yeasts (in an embodiment, the viability of the majority of the propagated yeasts) has been reduced by mechanical means (high pressure homogenization, bead beating, etc.), thermal means, chemical means (reducing agents, detergents, etc.) and/or enzymatic means. As such, the present disclosure provides a partially inactivated yeast product.

The process can also include a drying step. The drying step can include, for example, with roller-drying, electrospray-drying, spray-drying and/or fluid-bed drying.

The process can include providing additional products, as such, it may be necessary to further separate the components of the lysed recombinant yeast host cells. For example, the cellular wall components (referred to as a “insoluble fraction”) of the lysed recombinant yeast host cell may be separated from the other components (referred to as a “soluble fraction) of the lysed recombinant yeast host cells. This separating step can be done, for example, by using centrifugation and/or filtration. In some embodiments, the insoluble fraction is submitted to a washing step after separation. In some embodiments, the insoluble traction is submitted to a washing step prior to drying.

In some embodiments, the heterologous or native polypeptides can be provided as a yeast product (e.g., a product comprising a propagated yeast or a component of a propagated yeast). In the yeast product, the yeast can be provided as an inactive form. The yeast product can be provided in a liquid, semi-liquid, solid or dry form. In some embodiments, the yeast product is substantially free from the components of the propagated recombinant yeast host cell. In a specific embodiment, the yeast product is substantially free from the deoxyribonucleic acids from the propagated recombinant yeast host cell.

In alternative embodiments, the heterologous or native polypeptides can be provided as isolated (or substantially isolated) polypeptides.

### Methods of Using the Recombinant Yeast Host Cells for Making a Fermented Product

The recombinant yeast host cells of the present disclosure can be used in a method for making a fermented product from a biomass. This can be especially useful when the recombinant yeast host cells is capable of expressing, as a first genetic modification, one or more polypeptide capable of hydrolyzing, at least in part, the biomass being fermented (and optionally being liquefied). In such methods, the recombinant yeast host cells, optionally with fermenting yeasts, are contacted with the biomass under conditions so as to obtain an hydrolyzed liquefaction medium and/or a fermentation product. Fermented products, such as alcohols intended to be used biofuels, can be often obtained from fermenting a biomass with a yeast. The fermented product can be an alcohol, such as, for example, ethanol, isopropanol, n-propanol, 1-butanol, methanol, acetone and/or 1, 2 propanediol. In an embodiment, the fermented product is ethanol. Exemplary biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses, or sugar cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and proline-rich proteins). In some embodiments, the substrate comprises starch (in a gelatinized or raw form). In some additional embodiments, the biomass comprises or is derived from corn.

The fermentation step includes contacting a fermenting yeast with the mash or the raw biomass and maintaining this contact under conditions allowing the conversion of the biomass into the fermentation product. In the methods of the present disclosure, the recombinant yeast host cells can be considered as a fermenting yeasts. Optionally or in combination, the recombinant yeast host cells can be used in a co-culture with fermenting yeasts during the fermentation step. The present disclosure thus provides a fermented mash comprising the recombinant yeast host cells of the present disclosure or components thereof. The fermenting yeast can be, for example, from the genus Saccharomyces, Kluyveromyces, Arxula, Debaryomyces, Candida, Pichia, Phaffia, Schizosaccharomyces, Hansenula, Kloeckera, Schwanniomyces, Torula or Yarrowia. Suitable yeast species can include, for example, S. cerevisiae, S. bulderi, S. barnetti, S. exiguus, S. uvarum, S. diastaticus, S. boulardii, C. utilis, K. lactis, K. marxianus or K. fragilis. In some embodiments, the fermenting yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some further embodiments, the fermenting yeast is of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe or Schwanniomyces occidentalis. In one particular embodiment, the fermenting yeast is Saccharomyces cerevisiae. In some embodiments, the fermenting yeast can be an oleaginous yeast cell. For example, the oleaginous yeast cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiment, the fermenting yeast can be an oleaginous microalgae host cell (e.g., for example, from the genus Thraustochytrium or Schizochytrium). In an embodiment, the fermenting yeast is from the genus Saccharomyces and, in some embodiments, from the species Saccharomyces cerevisiae.

The fermentation step can be a batch-fed fermentation, a continuous fermentation or a combination of a plurality of fermentation cycles.

In an embodiment, the recombinant yeast host cells of the present disclosure are submitted to a plurality of fermentation cycles. The plurality of fermentation cycles in which the recombinant yeast host cells can be submitted comprises at least two distinct fermentation cycles: an initial fermentation cycle and one or more further fermentation cycles. In the initial fermentation cycle, a fermenting population comprising the recombinant yeast host cells (optionally in combination a population of fermenting yeasts) is contacted with a fermentation medium under conditions so as to obtain a fermentation product (and concurrently a fermented medium). The fermenting population obtained at the end of this initial fermentation cycle is recycled for a further fermentation cycle (e.g., substantially isolated and used to inoculate a further fermentation medium). It is recognized that the fermenting population used to inoculate the further fermentation medium can include contaminating wild yeasts which may have been introduced in the fermentation medium of the initial fermentation cycle. The inoculated further fermentation medium is then placed under conditions so as to obtain the fermented product and subsequently substantially isolate a (further) fermenting population (from a further fermented medium). The substantially isolated further fermenting population can be recycled and used to conduct one or more further fermentation cycle.

The fermentation step for making the fermented product can be performed at temperatures of at least about 20° C., about 21° C., about 22° C., about 23° C., about 24° C., about 25° C., about 26° C., about 27° C., about 28° C., about 29° C., about 30° C., about 31° C., about 32° C., about 33°, about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C.

In some embodiments, the fermentation step can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, at least about 500 mg per hour per liter, at least about 600 mg per hour per liter, at least about 700 mg per hour per liter, at least about 800 mg per hour per liter, at least about 900 mg per hour per liter, at least about 1 g per hour per liter, at least about 1.5 g per hour per liter, at least about 2 g per hour per liter, at least about 2.5 g per hour per liter, at least about 3 g per hour per liter, at least about 3.5 g per hour per liter, at least about 4 g per hour per liter, at least about 4.5 g per hour per liter, at least about 5 g per hour per liter, at least about 5.5 g per hour per liter, at least about 6 g per hour per liter, at least about 6.5 g per hour per liter, at least about 7 g per hour per liter, at least about 7.5 g per hour per liter, at least about 8 g per hour per liter, at least about 8.5 g per hour per liter, at least about 9 g per hour per liter, at least about 9.5 g per hour per liter, at least about 10 g per hour per liter, at least about 10.5 g per hour per liter, at least about 11 g per hour per liter, at least about 11.5 g per hour per liter, at least about 12 g per hour per liter, at least about 12.5 g per hour per liter, at least about 13 g per hour per liter, at least about 13.5 g per hour per liter, at least about 14 g per hour per liter, at least about 14.5 g per hour per liter or at least about 15 g per hour per liter. Ethanol production can be measured using any method known in the art. For example, the quantity of ethanol in fermentation samples can be assessed using HPLC analysis. Many ethanol assay kits are commercially available that use, for example, alcohol oxidase enzyme-based assays.

In some embodiments, the fermenting step is conducted under anaerobic conditions. As described above, yeast tends to undergo fermentation processes while under anaerobic conditions, while it tends to undergo propagation processes while under aerobic conditions. As used herein, “anaerobic conditions” means that the biomass is under an oxygen-poor environment. An oxygen-poor environment may have an oxygen concentration below that of air. For example, the concentration of oxygen may be below 21%, 20%, 15%, 10%, 9%, 8%, 7%, 6%, 5%, 4%, 3%, 2% or 1′)/0 by volume.

The methods of the present disclosure can also include a step of separating the fermentation product from other components of the fermented biomass. The methods of the present disclosure can also include a step of obtaining distillers' grains from the fermented biomass.

Optionally, a preliminary liquefaction step can be conducted to hydrolyze, at least in part, the starch molecules which are present, prior to the fermentation step. In the methods of the present disclosure, the recombinant yeast host cells can be introduced prior to, during and/or after the liquefaction step. A biomass which has been submitted to a liquefaction step can be referred to an hydrolyzed liquefaction medium (which can be an hydrolyzed slurry). The present disclosure thus provides an hydrolyzed liquefaction medium comprising the recombinant yeast host cells described herein or components thereof.

In the liquefaction step, the recombinant yeast host cells can be contacted with the liquefaction medium (which can be a slurry) under a condition to generate an hydrolyzed liquefaction medium (which can be an hydrolyzed slurry). In some embodiments, the recombinant yeast host cells are contacted with a liquefaction medium or a slurry which has not been submitted to a heat treatment step (and in some embodiments which is not intended to be submitted to a heat treatment step). In such instances, the recombinant yeast host cells are contacted with an untreated liquefaction medium or an untreated slurry under a condition so as to generate the hydrolyzed liquefaction medium. In some embodiments, the recombinant yeast host cells are contacted with a liquefaction medium or a slurry which has not yet been submitted to a heat treatment step but is intended to be submitted to such heat treatment step. In such instances, the recombinant yeast host cells are contacted with an untreated liquefaction medium or untreated slurry prior to the heat treatment step. In other embodiments, the recombinant yeast host cells are contacted with a liquefaction medium or a slurry which has already been submitted to a previous heat treatment step. In such instances, the recombinant yeast host cells are contacted with a gelatinized liquefaction medium or gelatinized slurry as the heat treatment would have favored at least partial disruption of the starch molecules which are present in the raw liquefaction medium/raw slurry (to provide a gelatinized liquefaction medium/slurry). In some embodiments, the contact between the gelatinized liquefaction medium and the recombinant yeast host cells can occur during the heat treatment step (at least in part). In some embodiments, the recombinant yeast host cells can be added to the liquefaction medium in the liquefaction process prior to, during and/or after a heat treatment has been applied.

As indicated herein, the liquefaction process can be performed entirely on an untreated liquefaction medium. However, in some embodiments, it may be advantageous to include a heat treatment step to the liquefaction process to liquefy, at least in part, a liquefaction medium comprising gelatinized starch molecules. The heat treatment step can improve the conversion of the starch molecules into dextrins and/or can reduce the time required to complete the liquefaction. The heat treatment step can include submitting the liquefaction medium (which may or may not include the enzyme combination) to a liquefaction temperature and for a liquefaction time period. In some embodiments, the liquefaction of starch occurs in the presence of recombinant microbial host cells and/or the microbial product described herein.

In some embodiments, the liquefaction temperature is at least about 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 95° C., 100° C., 105° C. or more can be used. In some further embodiments, the liquefaction temperature is between about 60° C. to 85° C. In some further embodiments, the liquefaction temperature is between about 70° C. to 75° C. In some further embodiments, the liquefaction temperature is between about 80° C. to 85° C. When the liquefaction temperature is between about 60° C. to 85° C. it can be maintained for a liquefaction time of about 60 minutes or more.

In some additional embodiments, a jet cooker can be used to provide the heat treatment step. In such embodiments, the liquefaction temperature can be at least about 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C. or more. Still in such embodiment, the liquefaction temperature can be maintained for a liquefaction time of about 1 minute or more.

The present invention will be more readily understood by referring to the following examples which are given to illustrate the invention rather than to limit its scope.

### EXAMPLE I—EXPRESSION OF HETEROLOGOUS PHOSPHOLIPASE

A phospholipase from Fusarium oxysporum was integrated into two distinct Saccharomyces cerevisiae transformants/strains. The nucleic acid molecule integrated in the yeast host cells encoded a phospholipase having an amino acid sequence of SEQ ID NO: 1, and was expressed with a signal sequence having amino acid sequence of SEQ ID NO: 2 (first transformant/strain) or 3 (second transformant/strain).

TABLE 1
Heterologous phospholipase sequences

| Sequence | | |
|---|---|---|
| Number | Description | Amino Acid Sequence |
| SEQ ID | Fusarium | SPVALDDYVNSLEERAVGVTTTDFSNFKFYIQHGAAAYCNSE |
| NO: 1 | oxysporum | AAAGSKITCSNNGCPTVQGNGATIVTSFVGSKTGIGGYVATDS |
| | phospholipase | ARKEIVVSFRGSINIRNWLTNLDFGQEDCSLVSGCGVHSGFQ |
| | | RAWNEISSQATAAVASARKANPSFNVISTGHSLGGAVAVLAA |
| | | ANLRVGGTPVDIYTYGSPRVGNAQLSAFVSNQAGGEYRVTHA |
| | | DDPVPRLPPLIFGYRHTTPEFWLSGGGGDKVDYTISDVKVCE |
| | | GAANLGCNGGTLGLDIAAHLHYFQATDACNAGGFSWRRYRS |
| | | AESVDKRATMTDAELEKKLNSYVQMDKEYVKNNQARS |
| SEQ ID | Hybrid | MRQVWFSWIVGLFLCFFNVSSAAPVNTTTEDETAQIPAEAVIG |
| NO: 2 | OST1/aMF | YSDLEGDFDVAVLPFSNSTNNGLLFINTTIASIAAKEEGVSLEK |
| | signal | REAEA |
| | sequence | |
| SEQ ID | Native signal | MLLLPLLSAITLAVA |
| NO: 3 | sequence of | |
| | Fusarium | |
| | oxysporum | |
| | phospholipase | |

The growth rate was determined to characterize the effect of phospholipase expression. The growth rate was determined at 30° C. in a YPD medium supplemented with 40 g/L of glucose.

When phospholipase expression was induced in the transformants, there was reduced growth regardless of the signal sequence used (see Table 2). For investigating the effect of phospholipase expression with different signal sequences, the growth rate was determined at 30° C. in a synthetic defined yeast uracil-dropout medium with 20 g/L glucose.

TABLE 2
Cell density of different cultures, after 24 hours of growth
(initial cell density ~0.1), with or without phospholipase
(PL) expression, and with different signal sequences.
With the signal sequence of SEQ ID NO: 3

| With PL expression induced | | PL expression uninduced | |
|---|---|---|---|
| Two sibling transformants | | Two sibling transformants | |
| 0.8576 | 0.8672 | 4.2736 | 4.3088 |

With the signal sequence of SEQ ID NO: 2

| With PL expression induced | | PL expression uninduced | |
|---|---|---|---|
| Two sibling transformants | | Two sibling transformants | |
| 3.5392 | 3.5024 | 4.8304 | 4.8048 |

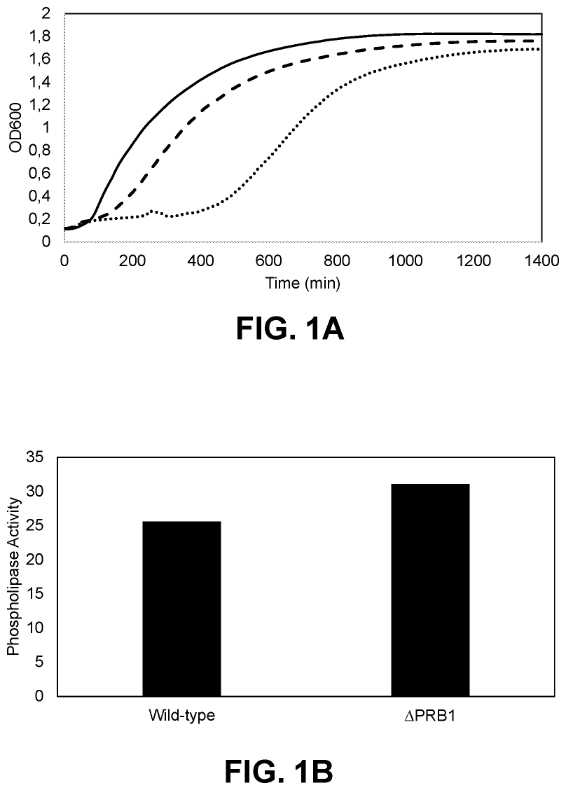

This is also shown in FIG. 1A, in which the engineered phospholipase expressing Saccharomyces cerevisiae stain (phospholipase expression strain) had a significantly lower growth rate as compared to a wild-type Saccharomyces cerevisiae stain. Hence, the introduction of heterologous phospholipase impaired the growth rate of the yeast host cell.

### EXAMPLE II—DELETION OF PRB1, VPS5, AND VPS17 IN HETEROLOGOUS PHOSPHOLIPASE EXPRESSING YEAST HOST CELL

To reduce the toxicity and growth rate impairments from expressing a heterologous phospholipase, the strain described in Example I expressing phospholipase having amino acid sequence of SEQ ID NO: 1 with signal sequence having amino acid sequence of SEQ ID NO: 2 were modified to delete certain native genes encoding proteins involved in the secretory pathway.

The growth rate of the modified strain was determined at 30° C. in a YPD medium supplemented with 40 g/L of glucose (YPD. 40).

FIG. 1A shows that by deleting the PRB1 gene (by removing the open reading frame from start codon to stop codon of the PRB1 gene which encodes vacuolar proteinase B), the growth can be significantly improved while still maintaining the phospholipase activity levels in the culture supernatant (compare the growth rate of the “phospholipase expression strain” with the growth rate of the “phospholipase expression strain with APrb1”). With a PRB1 deletion, the engineered Saccharomyces cerevisiae strain expressing phospholipase showed significantly improved growth rate, compared to the engineered phospholipase expression Saccharomyces cerevisiae strain but with unmodified PRB1 gene (see FIG. 1A).

In addition, as shown on FIG. 1 B, the deletion of PRB1 did not reduce phospholipase production by the engineered Saccharomyces cerevisiae strain, and instead slightly increased the phospholipid activity levels in the culture supernatant suggesting improved production level of phospholipase by the engineered Saccharomyces cerevisiae strain compared to a wild-type strain. Phospholipase activity was measured using Ped-Al (N-((6-(2,4-DNP)Amino)Hexanoyl)-1-(BODIPY™ FL C5)-2-Hexyl-Sn-Glycero-3-Phosphoethanolamine) as the substrate. The enzymatic reaction was run at 25° C., pH 5 for 10 min. The increase in relative fluorescent unit (ARFU) was determined which represents the apparent enzyme activity, which in turn was used to determine the amount of the active enzyme in the supernatant.

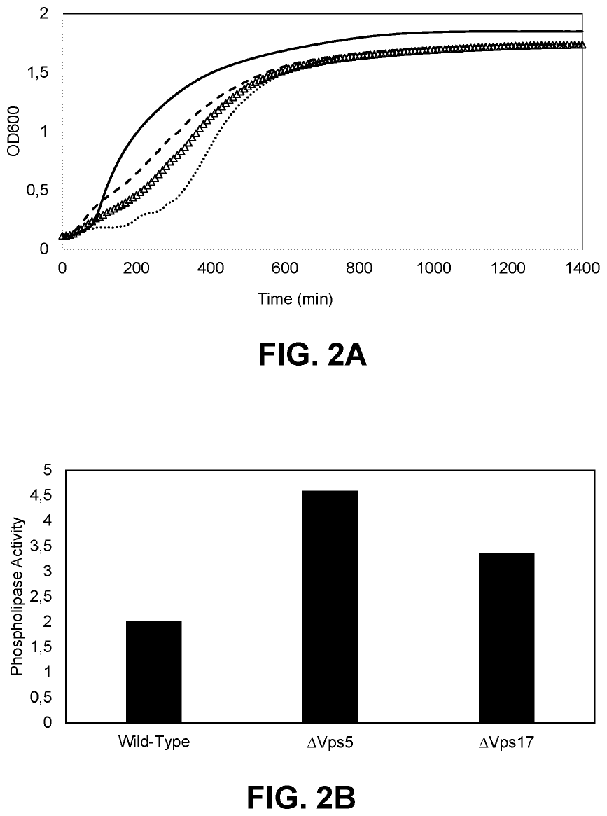

VPS5 and VPS17 are two proteins in the retromer complex of Saccharomyces cerevisiae that is responsible for trafficking proteins from prevacuolar or late endosome compartment back to the trans Golgi network. FIGS. 2A and 2B show that deletion of VPS5 or VPS17 (by removing their respective open reading frame from start codon to stop codon) improved the growth rate of an engineered phospholipase expressing Saccharomyces cerevisiae strain compared to one without the deletions (see FIG. 2A — compare the growth rate of the “phospholipase expression strain” with the growth rate of the “phospholipase expression strain with AVps5” or “phospholipase expression strain with AVps17”). As well, the VPS5 and VPS17 deletions also improved the secretion or production levels of phospholipase by the engineered Saccharomyces cerevisiae strain compared to a wild-type strain (see FIG. 2B).

### EXAMPLE III—OVEREXPRESSION OF NATIVE YEAST CHAPERONES IN HETEROLOGOUS PHOSPHOLIPASE EXPRESSING YEAST HOST CELL

The formation of inter- and intramolecular disulfide bonds is a crucial step in the folding of secreted proteins. Three ER chaperones, ERO1 (ER oxidase 1), PDI1 (Protein disulfide isomerase) and KAR2 (KARyogamy 2) have been reported to be involved in this process. Hence, overexpression of ERO1, PDI1 and KAR2 were investigated on the engineered Saccharomyces cerevisiae strains. The expression cassettes of ERO1, PDI1 and KAR2 (both derived from S. cerevisiae) were introduced to a locus that is not the native locus of ERO1 or PDI1 (at a neutral integration site). Promoters and terminators were used that are not native to ERO1 or PDI1, but are native Saccharomyces cerevisiae promoters/terminators. One copy of each gene was introduced per chromosome, for a total of 3 copies of each gene per cell (the yeast host cell was a triploid).

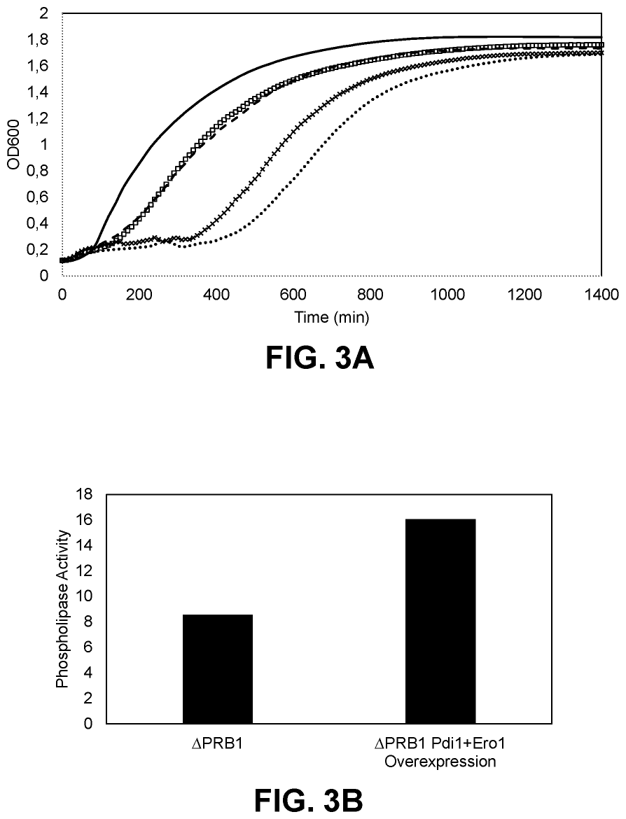

FIG. 3A shows that overexpressing PDI1 and ERO1 simultaneously in an engineered phospholipase expressing Saccharomyces cerevisiae strain improved the growth rate compared to the engineered phospholipase expressing Saccharomyces cerevisiae strain without the overexpression (compare the growth rate of the “phospholipase expression strain” with the growth rate of the “phospholipase expression strain with overexpression of Pdi1 and Erol”).

With PDI1 and ERO1 simultaneous overexpression together with PRB1 deletion the engineered phospholipase expressing Saccharomyces cerevisiae strain significantly improved not only its growth rate, but also the secretion level of phospholipase (see FIG. 3B).

### EXAMPLE IV—EFFECT ON GROWTH RATES

The maximum growth rates of the S. cerevisiae strains engineered in Examples II and III was determined during the exponential growth phase of cells grown in YPD 40 . It was then compared to the wild-type strain that do no expressed the phospholipase. The results are shown in Tables 5 3 and 4.

TABLE 3
Maximal growth rate of strains described in Example II. WT = wild-type,
PL = phospholipase, ΔVPS5 = inactivation of the VPS5 genes,
ΔVPS17 = inactivation of the VPS17 genes.

| | | Control = | WT + | WT + |
|---|---|---|---|---|
| Strain | WT | WT + PL | ΔVPS5 + PL | ΔVPS17 + PL |
| Growth rate (h⁻¹) | 1.47 | 0.5 | 1.1 | 0.77 |
| Growth compared | 100.0% | 34.0% | 74.8% | 52.4% |
| to WT | | | | |
| Growth compared | N. D. | 100% | 220% | 154% |
| to control | | | | |

TABLE 4
Maximal growth rate of strains described in Example III.

| | | | WT + | WT + | WT + ΔPRB1 + | WT + ERO1 + |
|---|---|---|---|---|---|---|
| | | Control = | ΔPRB1 + | ERO1 + | ERO1 + | PDI1 + |
| Strain | WT | WT + PL | PL | PDI1 + PL | PDI1 + PL | KAR2 + PL |
| Growth rate (h⁻¹) | 1.02 | 0.37 | 0.75 | 0.46 | 0.85 | 0.84 |
| Growth compared | 100.0% | 36.3% | 73.5% | 45.1% | 83.3% | 82.4% |
| to WT | | | | | | |
| Growth compared | N.D. | 100% | 203% | 124% | 230% | 227% |
| to control | | | | | | |

WT = wild-type, PL = phospholipase, ΔPRB1 = inactivation of the PRB1 genes, ERO1 = overexpression of the ERO1 gene, PDI1 = overexpression of the PDI1 gene, KAR2 = overexpression of the KAR2 gene.

### EXAMPLE V—EXPRESSION OF HETEROLOGOUS FUMONISIN ESTERASE

One or more copies of a heterologous fumonisin esterase gene was integrated in Saccharomyces cerevisiae strains and its effect on the growth rate of the corresponding recombinant yeast host cells was determined. It was also determined if the introduction of additional genetic modifications could modulate the growth rate of the corresponding recombinant yeast host cells. Table 5 provides the genotype of the various strains tested in this Example.

Table 5. Genotype of the Saccharomyces cerevisiae strains presented in this Example. FE3 refers to a heterologous gene encoding a secreted polypeptide having fumonisin esterase activity of SEQ ID NO: 21. The expression cassette for the expression of FE3 also included the signal sequence from the alpha mating factor 1 gene to allow for the secretion of the polypeptide and which was subsequently cleaved after secretion. PDI1 (Protein disulfide isomerase 1) refers to the presence of a further copy of the S. cerevisiae gene encoding the PDI1 polypeptide. ERO1 (ER oxidase 1) refers to the presence of a further copy of the S. cerevisiae gene encoding the ERO1 polypeptide.

| Designation | Heterologous gene(s) | Inactivated native gene(s) |
|---|---|---|
| M3836 (WT) | Selection marker | hoΔ |
| M24868 | 1 copy of FE3 | hoΔ |
| | Selection marker | fcy1Δ |
| M26599 | 3 copies of FE3 | hoΔ |
| | | fcy1Δ |
| M27750 | 5 copies of FE3 | hoΔ |
| | | fcy1Δ |
| | | ime1Δ |
| M28728 | 5 copies of FE3 | hoΔ |
| | Selection marker | fcy1Δ |
| | | ime1Δ |
| | | vps5Δ |
| M29002 | 5 copies of FE3 | hoΔ |
| | | fcy1Δ |
| | | ime1Δ |
| | | prb1Δ |
| M29004 | 5 copies of FE3 | hoΔ |
| | | fcy1Δ |
| | | ime1Δ |
| | | vps5Δ |
| M29005 | 5 copies of FE3 | hoΔ |
| | | fcy1Δ |
| | | ime1Δ |
| | | vps17Δ |
| M29019 | 5 copies of FE3 | hoΔ |
| M29020 | 1 copy of PDI1 | fcy1Δ |
| | 1 copy of ERO1 | ime1Δ |
| | | prb1Δ |
| M29021 | 5 copies of FE3 | hoΔ |
| M29022 | 1 copy of PDI1 | fcy1Δ |
| | 1 copy of ERO1 | ime1Δ |
| | | vps5Δ |
| M29023 | 5 copies of FE3 | hoΔ |
| | 1 copy of PDI1 | fcy1Δ |
| | 1 copy of ERO1 | ime1Δ |
| | | vps17Δ |
| M29028 | 5 copies of FE3 | hoΔ |
| M29029 | Selection marker | fcy1 |
| | | ime1Δ |
| | | vps5Δ |
| | | prb1Δ |

Each of the strains, initially were grown in patches on YPD₄₀ plate, were inoculated in 5 mL of YPD₄₀ and grown overnight, under agitation, at 35° C. The overnight cultures (in triplicates for each strain) was normalized to OD=1, diluted to 1:1000 and grown in YP₁₀ medium at 35° C. for 72 h. Cell growth was determined using optical density at 600 nm using a Spectramax™ microplate reader. The maximal growth rate and time to reach OD 0.3 were determined for each strain and is presented in Table 6. The time to reach OD 0.3 is impacted by the growth rate as well as the lag time.

TABLE 6
Maximal growth rate of the strains presented in table 5

| Designation | Maximal growth rate (h⁻¹) | Time to OD 0.3 (hours:minutes) |
|---|---|---|
| M3836 | 0.40 | 10:40 |
| M24868 | 0.38 | 11:00 |
| M26599 | 0.23 | 13:30 |
| M27750 | 0.17 | 22:10 |
| M28728 | 0.19 | 20:00 |
| M29002 | 0.21 | 16:50 |
| M29004 | 0.18 | 18:10 |
| M29005 | 0.22 | 13:50 |
| M29019 | 0.17 | 12:40 |
| M29020 | 0.20 | 14:20 |
| M29021 | 0.17 | 16:30 |
| M29022 | 0.15 | 15:50 |
| M29023 | 0.24 | 13:20 |
| M29028 | 0.23 | 12:50 |
| M29029 | 0.18 | 15:20 |

As indicated in Table 6, the expression of the heterologous FE3 polypeptide reduced the maximal growth rate and time to reach OD 0.3 of the yeast (compare M3836 control strain not expressing the heterologous FE3 polypeptide with strains M24686 (one copy of the gene encoding FE3), M26599 (three copies of the gene encoding FE3) and M27750 (five copies of the gene encoding FE3).

The deletion of the native gene vps5 in FE3-expressing strains M28728 and M29004 did improve the growth rate, when compared to the FE3-expressing strain M27750 lacking such genetic modification (Table 6). The combination of the deletion of the native gene vps5 as well as the expression of the heterologous PDI1/ERO1 polypeptides in FE3-expressing strains M29021 and M29022 further improved time to reach OD 0.3, when compared to strains M28728 and M29004 (Table 6).

The deletion of the native gene vps17 in FE3-expressing strains M29005 did improve the growth rate and decreased the time to reach OD 0.3, when compared to the FE3-expressing strain M27750 lacking such genetic modification (Table 6). The combination of the deletion of the native gene vps17 as well as the expression of the heterologous PDI1/ERO1 polypeptides in FE3-expressing strain M29023 further improved the growth rate, when compared to strains M29005 e (Table 6).

The deletion of the native gene prb1 in FE3-expressing strain and M29002 did improve the growth rate and decrease the time to reach OD 0.3, when compared to the FE3-expressing strain M27750 lacking such genetic modification (Table 6). The combination of the deletion of the native gene prb1 as well as the expression of the heterologous PDI1/ERO1 polypeptides in FE3-expressing strains M29019 and M29020 further improved the growth rate, when compared to strain M29002 (Table 6).

The deletion of the native genes vps5 and prb1 in FE3-expressing strain M29028, and M29029 did improve the growth rate, when compared to the FE3-expressing strain M27750 lacking such genetic modifications (Table 6).

### EXAMPLE VI—EXPRESSION OF HETEROLOGOUS ALPHA-AMYLASE

One or more copies of a heterologous alpha-amylase gene was integrated in Saccharomyces cerevisiae strains and its expression on the growth rate of the corresponding recombinant yeast host cells was determined. It was also determined if the co-expression of PDI1 could also modulate the growth rate of the corresponding recombinant yeast host cells. Table 7 provides the genotype of the various transformants/strains tested in this Example.

TABLE 7
Genotype of the Saccharomyces cerevisiae strains presented in this Example. AA refers
to a heterologous gene encoding a secreted and tethered polypeptide having alpha-amylase
activity of SEQ ID NO: 22. PDI1 (Protein disulfide isomerase 1) refers to the presence of a further
copy of the S. cerevisiae gene encoding the PDI1 polypeptide (SEQ ID NO: 27).

| Designation | Heterologous gene(s) | Inactivated native gene(s) |
|---|---|---|
| M10580 | This is a wild-type strain | |
| T11203 | 1 copy of AA | Not applicable |
| T11204 | 1 copy of AA | Not applicable |
| | 1 copy of PD1 | |

Yeast culture conditions. A total of 4 individual yeast transformants T11203 and T11204 were grown in 600 μl YP-Sucrose (YPS) 20 g/L for 48 h in 96 well plates at 32° C. with 900 rpm shaking. The parental control, M10580, was grown in duplicates and inoculated from a patched YPD plate.

Growth rate determination. A 1:1000 dilution was made from the 48 h cultures into fresh YPS and a 100 μl transferred to a Falcon round-bottom 96 well plate and incubated at 32° C. in a Biotek Multitron plate reader with optical density measured every 10 mins over 48 h. The maximum specific growth rate (μmax) is the point in the fermentation at which the cells are growing at the highest specific growth rate as calculated by the equation:

μ=In(N(t)−N0)/t

wherein μ (h−1) is the specific growth rate and N(t) final cell density, N0 is the original cell density, and t is the time (in h) between samples.

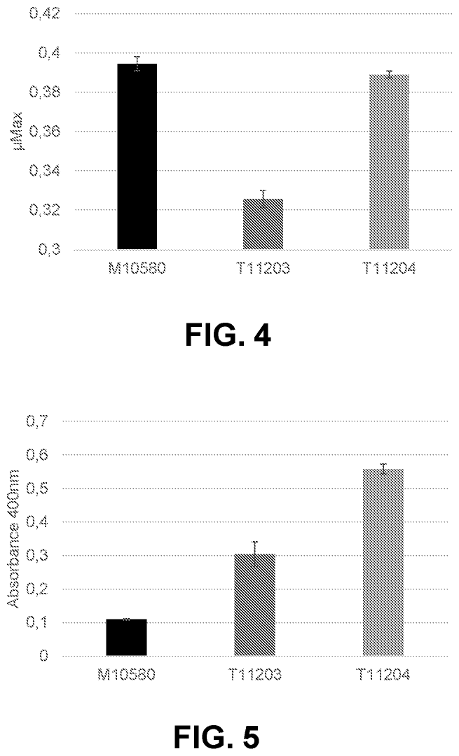

The expression of the heterologous alpha-amylase did reduce the maximal growth rate of the yeast expressing it (compare M10580 and T11203 on FIG. 4). However, the co-expression of the heterologous alpha-amylase as well as PDI1 did restore, at least in part, the maximal growth rate of the yeast expressing both polypeptides (compare T11204 and T11203 on FIG. 4).

Alpha-amylase activity determination (Ceralpha assay). A 150 μl aliquot of the culture was transferred to a fresh 200 μl 96 well plate and centrifuged at 3000 g for 5 mins. Secreted (supernatant) and cell-associated activity were independently evaluated using the Megazyme Cerlpha assay per the recommended protocol (Cat #K-Cera), with the following modifications. A 150 μl aliquot of the culture was transferred to a fresh 200 μl 96 well plate and centrifuged at 3000 g for 5 mins. The supernatant was removed and used in the assay with 10 μl of supernatant with 20 μl AMY HR reagent, incubated for 15 min at 50° C. The cell pellet was washed 2× with water and treated with 150 μl Y′Per extraction reagent (Thermo Scientific Cat #78991) for 20 mins at room temperature with 10 μl of the cell solution added to 20 μl AMY HR reagent and incubated for 30 mins at 50° C. As shown on FIG. 5, the alpha-amylase activity of T11204 was higher than the one associated with T11203.

### REFERENCES

Farquhar, R. et al. Protein disulfide isomerase is essential for viability in Sacchavomyces cerevisiae. 108, 81-89 (1991).

Frand, A. R. & Kaiser, C. A, The ERO1 gene of yeast is required for oxidation of protein dithiols in the endoplasmic reticulum, Mol Cell 1, 161-170 (1998).

Frauenlob, J., Scharl, M., D'Amico, S. & Schoenlechner, R. Effect of different lipases on bread staling in comparison with Diacetyl tartaric ester of monoglycerides (DATEM), Cereal Chem. 95, 367-372 (2018).

Goesaert, H. et al. Wheat flour constituents: How they impact bread quality, and how to impact their functionality, in Trends in Food Science and Technology 16, 12-30 (Elsevier, 2005).

Houde, A., Kademi, A, & Leblanc, D. Lipases and their industrial applications: an overview. Appl, Biochem, Biolechnol. 118, 155-70 (2004).

Huang, Mingtao, et al. “Engineering the protein secretory pathway of Saccharomyces cerevisiae enables improved protein production.” Proceedings of the National Academy of Sciences 115.47 (2018): E11025-E11032.

Kousuke Kuroda, Yoshinori Kitagawa, Kazuo Kobayashi, Haruhiko Tsumura, Toshihiro Komeda, Eiji Mori, Kazuhiro Motoki, Shiro Kataoka, Yasunori Chiba, Yoshifumi Jigami, Antibody expression in protease-deficient strains of the rnethylotrophic yeast Ogataea minuta, FEMS Yeast Research, Volume 7, Issue 8, December 2007, Pages 1307-1316

Marsalek, L., Puxbaum, V., Buchetics, M. et al. Disruption of vacuolar protein sorting components of the HOPS complex leads to enhanced secretion of recombinant proteins in Pichia pastoris. Microb Cell Fact 18, 119 (2019).

Morozkina E V, IViarchenko A N, Keruchenko J S, Keruchenko I D, Khotchenkov V P, Popov V O, Benevolensky S V. Proteinase B disruption is required for high level production of human mechano-growth factor in Saccharomyces cerevisiae. J Mol Microbiol Biotechnol, 2010;18(3)188-94,

Robinson, A., Hines, V. & Wittrup, K. Protein Disulfide isomerase Overexpression Increases Secretion of Foreign Proteins in Saccharomyces cerevisiae, Nat Biotechnol 12, 381-384 (1994).

de Ruijter J C, Koskela E V, Frey A D. Enhancing antibody folding and secretion by tailoring the Saccharomyces cerevisiae endoplasmic reticulum. Microb Cell Fact. 2016;15:87. Published 2016 May 23.

Smith J D, Tang B C, Robinson A S, Protein disulfide isomerase, but not binding protein, overexpression enhances secretion of a non-disulfide-bonded protein in yeast. Biotechnol Bioeng. 2004 Feb 5;85(3)

Wentz A E, Shusta E V. A novel high-throughput screen reveals yeast genes that increase secretion of heterologous proteins. Appl Environ Microhiol. 2007,73(4):11891198. doi:10.11281AEM .02427-06

Wu, M., Shen, Q., Yang, Y. et al. Disruption of YPS1 and PEP4 genes reduces proteolytic degradation of secreted HSA/PTH in Pichia pastoris GS115, J Ind Microbic Biotechnol 40, 589-599 (2013).

1. A recombinant yeast host cell having:
(i) a first genetic modification for (a) expressing a heterologous polypeptide or over-expressing a native polypeptide and (b) a signal sequence operatively associated with the heterologous polypeptide or the native polypeptide; and
(ii) a second genetic modification for increasing the growth rate of the recombinant yeast host cell when compared to the growth rate of a control yeast host cell;
wherein the control yeast host cell expresses the heterologous polypeptide or over-expresses the native polypeptide and lacks the second genetic modification; and
wherein the expression of the heterologous polypeptide or the over-expression of the native polypeptide impedes the growth rate of the control yeast host cell when compared to a parental yeast host cell.

2. (canceled)

3. The recombinant yeast host cell of claim 1, wherein the first heterologous nucleic acid molecule or the first native nucleic acid molecule is operatively associated with a propagation or an aerobic promoter.

4.-5. (canceled)

6. The recombinant yeast host cell of claim 1, wherein the heterologous or native polypeptide has phospholipase activity, fumonisin esterase activity or a alpha-amylase activity.

7. The recombinant yeast host cell of claim 6, wherein:
the polypeptide having phospholipase activity comprises an amino acid sequence of SEQ ID NO. 1 or 4, is a variant of the amino acid sequence of SEQ ID NO: 1 or 4 having phospholipase activity, or is a fragment of the amino acid sequence of SEQ ID NO: 1 or 4 having phospholipase activity;
the polypeptide having fumonisin esterase activity comprises an amino acid sequence of SEQ ID NO: 21, is a variant of the amino acid sequence of SEQ ID NO: 21 having fumonisin esterase activity, or is a fragment of the amino acid sequence of SEQ ID NO: 21 having fumonisin esterase activity; or
the polypeptide having alpha-amylase activity comprises an amino acid sequence of SEQ ID NO: 23, is a variant of the amino acid sequence of SEQ ID NO: 23 having alpha-amylase activity, or is a fragment of the amino acid sequence of SEQ ID NO: 23 having alpha-amylase activity.

8. (canceled)

9. The recombinant yeast host cell of claim 1, wherein the second genetic modification:
is a modification in a yeast protein secretory and trafficking pathway;
comprises the modulation in the expression of a gene associated with a redox enzyme;
comprises the modulation in the expression of a gene involved in endoplasmic reticulum associated degradation pathway (ERAD);
comprises the modulation in the expression of a gene involved in endoplasmic reticulum (ER) expansion;
comprises the modulation in the expression of a gene involved in translocation to the endoplasmic reticulum (ER);
comprises the modulation in the expression of a gene involved in polypeptide trafficking from the ER to the Golgi system, and/or from the Golgi system to the plasma membrane;
comprises the modulation in the expression of a gene involved in polypeptide trafficking from the Golgi system to the plasma membrane;
comprises the modulation in the expression of a gene involved in the vacuolar sorting pathway;
comprises the modulation in the expression of a gene associated with a polypeptide trafficking toward vacuoles;
comprises the modulation in the expression of a gene associated with a cell wall mannoprotein;
comprises the modulation in the expression of a gene involved in transcription and/or translation;
comprises the modulation in the expression of a gene associated with a ribosome; and/or
comprises the modulation in the expression of a gene associated with a protease involved in the activation of proproteins of the secretory pathway.

10. The recombinant yeast host cell of claim 9, wherein the second genetic modification is:
one or more of a modification of a yeast translation pathway;
one or more of a modification of a yeast post-translational modification pathway;
one or more of modification of a yeast protein trafficking pathway;
one or more of modification of a yeast vacuolar pathway;
one or more of a modification of a yeast cell wall stability pathway; or
combinations thereof.

11. The recombinant yeast host cell of claim 10, wherein the second genetic modification comprises a modulation in the expression of:
a gene involved in polypeptide folding, glycosylation and degradation in the endoplasmic reticulum (ER);
a gene associated with an unfolded protein response (UPR);
a PDI1 gene, an ERO1 gene, and/or a CPR5 gene;
a HTM1 gene, a YOS9 gene, a HDR1 gene, a HDr3 gene, an UBC7 gene, and/or a DER1 gene;
an OPI1 gene, and/or a PAH1 gene;
a SIL1gene;
a gene associated with a COPII vesicle;
a gene associated with a COPI vesicle;
a gene associated with an endosome;
a SEC1 gene, a SEC4 gene, a SSO1 gene, a SSO2 gene, a SNC2 gene, an EXO70 gene, and/or a YPT32 gene;
a gene associated with a vacuolar protease;
a VPS8 gene, and/or a VPS21. gene;
a gene involved in autophagy;
a CCW12 gene, and/or a CWP2 gene;
a native SEDI gene;
a BMH2 gene, a HSF1 gene, a SRP14 gene, and/or a SRP54 gene;
a BFR2 gene, and/or a RPP0 gene; and/or
a KEX2 gene.

12. The recombinant yeast host cell of claim 11, wherein the second genetic modification comprises the modulation in the expression of a gene associated with a molecular chaperone.

13. The recombinant yeast host cell of claim 12, wherein the gene associated with the molecular chaperone comprises a KAR2 gene, a LHS1 gene, a JEM1 gene, a SSA1 gene, a SSA4 gene and/or a SSE1 gene.

14. (canceled)

15. The recombinant yeast host cell of claim 11, wherein:
the gene associated with the UPR comprises a HAC1 gene, an IRE1 gene, and/or a KIN2 gene;
the vacuolar protease is a PRB1 gene, a VMA3 gene, and/or a PEP4 gene.

16.-25. (canceled)

26. The recombinant yeast host cell of claim 11, wherein the gene associated with the COPII vesicle comprises an ERV25 gene, an ERV29 gene, a SAR1 gene, a SEC12 gene, a SEC13 gene, a SEC16 gene, a SEC23 gene, a SE24 gene, a SEC31 gene, a SBH1 gene, COG5 gene, a COG6 gene, a BOS1 gene, COY1 gene, and/or a SLY1 gene.

27. (canceled)

28. The recombinant yeast host cell of claim 11, wherein the gene associated with the COPI vesicle comprises a GOS1 gene, and/or a LAM1 gene.

29. (canceled)

30. The recombinant yeast host cell claim 11, wherein the gene associated with the endosome comprises a VPS5 gene, a VPS17 gene, a VPS26 gene, a VPS29 gene, and/or a VPS35 gene.

31.-38. (canceled)

39. The recombinant yeast host cell of claim 11, wherein the gene involved in autophagy comprises a MTC6 gene, and/or a SEC4 gene.

40.-49. (canceled)

50. The recombinant yeast host cell of claim 1, wherein the recombinant yeast host cell is from the genus Saccharomyces sp. and/or from the species Saccharomyces cerevisiae.

51. A process for propagating the recombinant yeast host cell of claim 1, the process comprising culturing the recombinant yeast host cell in a culture medium under conditions so as to allow the propagation of the recombinant yeast host cell.

52.-55. (canceled)
